(19) United States
(12) Patent Application Publication
(10) Pub. No.: US 2020/0211720 A1
(43) Pub. Date: Jul. 2, 2020

(54) SURGICAL MEDIA STREAMING, ARCHIVING, AND ANALYSIS PLATFORM

(71) Applicant: Engauge LLC, Boulder, CO (US)

(72) Inventor: Daniel Goldberg, Boulder, CO (US)

(21) Appl. No.: 16/732,000

(22) Filed: Dec. 31, 2019

Related U.S. Application Data
(60) Provisional application No. 62/787,091, filed on Dec. 31, 2018.

(51) Int. Cl. G16H 80/00; G16H 10/60; G16H 15/00; G06N 3/02; G06F 21/62

(52) U.S. Cl. CPC G16H 80/00; G16H 10/60; G06F 21/6245; G06N 3/02; G16H 15/00

(57) ABSTRACT

A surgical media management system for capture, real-time communication, and storage of surgical media data. The surgical media data may be received at a source computing device executing a source real-time communication (RTC) client. The source RTC client may communicate the surgical media data to a surgical media management platform which may also mediate a real-time peer to peer connection with a participant RTC client for a real-time broadcast of surgical media data. The surgical media management platform also stores the surgical media data in cloud storage for later retrieval and viewing. Furthermore, an analytics engine may be provided to analyze the surgical media data to determine actionable analytics regarding favorable outcomes of surgical procedures.

### CROSS-REFERENCE TO RELATED APPLICATIONS

The present application claims benefit of priority to U.S. Provisional Patent Application No. 62/787,091, entitled “A LOW COST MEDIA ARCHIVING PLATFORM, REAL-TIME INTERACTIVE MEDIA SESSION PLATFORM, AND PRIVATE/PUBLIC SOCIAL NETWORK PLATFORM, FOR SURGEON TRAINING, SURGEON PERFORMANCE IMPROVEMENT, PATIENT CARE, PATIENT SAFETY, ENHANCING HOSPITAL QUALITY, AND INCREASING VALUE FOR THE HEALTHCARE INSTITUTION” filed on 31 Dec. 2018, which is expressly incorporated by reference herein for all that it discloses or teaches.

### BACKGROUND

Healthcare providers faces serious issues that include, among others, attempting to deliver the highest quality of care, reducing the cost of healthcare, and maintaining or increasing government reimbursement levels for the delivered care. These concerns have created new impetus to develop and implement approaches to lower costs, pursue better patient outcomes, and secure the highest reimbursement. Government healthcare providers, private healthcare providers, and private healthcare insurance providers now look acutely at the quality of care delivered, patient outcomes, and the patient experience in an attempt to increase the quality of healthcare provided while attempting to reduce costs. In some contexts, requirements have been imposed that may mandate that healthcare providers (e.g., doctors, facilities, hospitals, or the like) report various outcomes and patient experience data related to the patient's encounter associated with the provision of healthcare.

In turn, an approach that may be referred to as “value based payments” may be implemented in which the performance of the healthcare provider and the experience of their patient (objective and/or subjective results) are tied directly to a quality assessment of the patient encounter. In turn, the quality assessment of the patient encounter may be associated with the amount of money that the healthcare provider will be paid or reimbursed for the care delivered. For instance, in the United States, all Medicaid Managed Care Organizations (MCOs) must transition to a value based payment structure for at least 80-90% of their provider payments. Reimbursements to healthcare providers from the government (e.g., under U.S. Medicare and/or Medicaid programs) are based on the quality assessment of the patient encounter that is based on patient outcome and patient experience data. In turn, payment reduction penalties are applied if certain negative data is reported, such as reduced patient experience, patient readmission, surgical site infections after surgery, or other negative outcomes or experiences. Thus, hospitals are incentivized to achieve high levels of value for their work through the government scrutinized level of quality, the patient's experience, and overall care. In turn, the migration to a value based payment structure creates new objectives and targets for healthcare providers.

Thus, beyond the core commitment to the best patient care, healthcare institutions face regulatory and/or market pressures to deliver value and quality associated with the provision of healthcare as measured in the value based payment system. In turn, the metric of value and quality of a healthcare provider is reflected in patient experience, patient readmission, surgical site infections after surgery, or other metrics that are used to evaluate healthcare providers and, at least to some degree, dictate reimbursement levels for care provided. In essence, the better a healthcare provider performs in relation to the quality assessment of the patient experience are the more such healthcare providers will be compensated for the care provided. This incentive is ultimately designed to increase patient outcomes by incentivizing higher performing healthcare providers, such as surgeons.

To this end, technology tools can profoundly aid a healthcare provider in monitoring, evaluating, and managing the provision of healthcare in a value based payment structure. However, such technologies may be difficult to implement given the scarcity of information technology (IT) personnel and IT budget funds to acquire and/or maintain such new technologies. This is in large part due to the burden placed on IT departments and their budgets as they are enmeshed with the purchase and implementation of ongoing technology projects pursuant of regulatory measures and acute IT security concerns, such as Health Insurance Portability and Accountability Act (HIPAA) compliance, patient data privacy, International Statistical Classification of Diseases and Related Health Problems (ICD10) medical classification, Electronic Health Records (EHR), and other various IT security product implementations. Thus, a fundamental tension exists with the adoption and implementation of new technologies that can also benefit the care provider because the existing teams and budgets are overtaxed by these and other endeavors. Thus, resources are generally limited or unavailable for implementing new technologies benefitting the healthcare provider in a value based payment system. The costs alone of these technologies also create a challenge for the healthcare provider to allocate funds for the needed technologies. As such, only a product at a profoundly lower cost that technologies is feasible in view of the challenges faced today at healthcare providers worldwide.

One example of new technology implemented in relation to the delivery of healthcare is the use of advanced media (e.g., video) in surgery, which has become a valuable tool for surgeons. In nearly every surgical specialty type today (general surgery, gynecology, neurology, thoracic surgery, etc.), surgical decision making is executed through video-based efforts. This generally means that the surgeon uses a video device to see inside the patient. The video acquisition may involve endoscopic, laparoscopic, arthroscopic, hysteroscopic, video-microscopic, and/or other methods with which the surgeon and the surgical team watch closely and usually entirely (without open surgical wound exposure to the anatomy for a direct view without the video source) in order to make the surgical decisions. Such video is of a high definition (HD) quality, and the content captures subtle details of the actions of the surgeon, the anatomy of the patient, and/or the actions of the devices employed. In short, such videos, in effect, “tell the whole story” of the surgery. It is estimated that approximately 50 million surgical videos are generated in U.S. hospitals each year.

A preserved record of what transpires in the operating theatre is precious and invaluable not only for the patient, but also for other patients, surgeons, and/or surgical trainees to benefit from. Use of video tools in the training of personnel allow practitioners beyond the operating surgeon (e.g., including doctors, nurses, surgeons, hospital administration, etc.) to learn from what is encountered in the operating theatre by others. The ability to use video and audio recordings of a surgery (e.g., surgical media data) increases the number of persons that may gain knowledge from a surgical experience by observing the surgical media data and interacting with it. The collective benefit from observing a surgery may range from students that use the surgical media data to learn to colleagues who can assist the active surgery. However, physically attending a surgery creates a negative impact on the operational efficiencies of the operating theater, which can impair the surgical team's focus and, thus, create patient safety concerns. In this regard, heretofore the ability to share the experience of observing a surgery to “deliver” the surgical experience beyond the walls of the operating theater has required technologically sophisticated tools at a high cost that are not operationally efficient or cost effective for a healthcare provider.

While medical device and imaging device providers have spent years refining multimedia (e.g., video capture) products and have in certain instances offered legacy, off-line means of recording the surgical video locally in the operating rooms, these approaches have limitations that have limited the adoption and implementation of such solutions. For example, conventional approaches often involved non-encrypted and unmanaged ways of recording, transporting, and sharing the videos. In this regard, a surgeon may make a quick copy of the video onto a non-encrypted jump drive and leave the operating room with that device. The use of such a device is not compliant with conventional healthcare IT security regulations and governmental regulations regarding patient privacy. If the device were lost, governed protected health information is unencrypted and available for malicious or untoward use. Some governments issue fines for such mishandling of protected health information. Moreover, without centralized and managed systems to archive and structure the video data, the healthcare institution loses the value intrinsic in the surgical video, which provides value for training other surgeons, value for medical reasons, value for legal reasons, and value for insurance or reimbursement reasons, and a value in evaluating the surgeon's performance. Lastly, without a managed system, the highly unavailable and highly cost billed surgeon loses precious time wrangling their videos from drive to drive, room to room every day.

Accordingly, while most surgeons find great value in the surgical videos they record, these providers have not been offered simple, low cost, regulatory compliant, and IT-friendly tool to manage the surgical video so they can leverage them for their various professional, educational and healthcare institution interests.

### SUMMARY

Based on the foregoing the present disclosure is generally directed to a surgical media management platform that facilitates a cost-effective, regulatory compliant, user-friendly, and analytically powerful approach to the management of surgical media data. The platform includes straightforward tools that allow surgical media data to be captured in real-time for storage, distribution, and/or analysis. Moreover, despite the simplicity and operational efficiencies of the surgical media management platform, the platform provides strict regulatory compliance through advanced encryption and other security protocols for the secure, centrally managed storage, sharing, and/or analysis of the surgical media data captured in the system. In turn, surgical media data may be more easily leveraged to provide training, operational insights, documentation, or other relevant purposes.

The platform, through the examples described herein, is designed to function to place the healthcare delivery team, and especially the surgeon, in a position to perform better while at the same time help the healthcare provider to achieve better outcomes, better patient experience measurement, and, generally, a higher value of care. This may directly translate into better patient outcomes, and in turn, higher revenue for healthcare providers in a value based payment system.

Moreover, the present disclosure also functions to enable medical device providers, whose interest is to innovate new products that impact care delivery and succeed in the marketplace with those products, to more quickly and more cheaply innovate their products and capture product adoption at an accelerated rate, with greater ease via connectivity to the operating theatre and its events. In turn, the platform described herein may allow medical device providers to provide innovative devices at a lower cost through efficient development, marketing and sales, and improved training and user adoption. In turn, the surgical medial data management platform described herein may be leveraged by medical device providers to improve development and product implementations, which may result higher profit and revenues for the medical device providers.

Specifically, the present disclosure generally describes systems and methods that can be employed to address one or more of the shortcomings recited above by recording a surgery using surgical media to extract and deliver surgical media data regarding the surgery beyond the walls of the operating theatre. Thus, the surgical experience documented in the surgical media data may be more easily shared in a regulatory compliant manner with interested stakeholders including trainees and healthcare facilities.

The present disclosure also facilitates the structuring of surgical media libraries based on the content of surgical media. Providing such a structured surgical media library may be leveraged in a number of ways to improve patient outcomes and provide valuable operational improvements to healthcare providers. For example, leveraging surgical media data stored in a structured library using advanced machine learning, computer modeling, and/or artificial intelligence may provide new, meaningful insights into surgeries to assist in improving patient outcomes and returning improved performance of a healthcare provider. In turn, the present disclosure facilitates innovative ways to meet the financial concerns, address a scarce labor resource environment, and provide valuable features and benefits for healthcare providers and medical device providers.

The present disclosure includes the capture, archiving, and live broadcasting of surgical media data (e.g., video and, at times, audio) from the operating theatre. The examples provided leverage straightforward hardware requirements by design to allow for user-friendly, computationally simple, and reliable means to extract the surgical media data from the operating theatre to make it available in an structured media archive or via a live media stream utilizing a surgical media management platform according to the present disclosure. Accordingly, the surgical media data management platform is used to connect existing video sources that are already present in the operating theatre through readily available and inexpensive hardware components (e.g., a personal computer placed in the operating theatre) to deliver surgical media data to the archiving and live broadcast platform. Given the simplicity of the hardware required for a surgeon or healthcare facility to avail themselves of the platform adoption and integration of the platform into existing systems, the advantages described above are highly achievable at profoundly lower costs than current solutions.

Specifically, the architecture employed by the platform can include a web-based or cloud-based infrastructure. Such infrastructure significantly lowers the total cost of the platform because it removes the need for local hospital IT team labor resources and removes the need for the hospital to purchase, install, and manage expensive hardware infrastructure.

Further still, a web-based system facilitates user-friendly, easy, and economic access to participants outside of healthcare facilities to view surgical media data. Whether it be a live broadcast or archived surgical media, the surgical media may be accessed by authorized users using any standard computing device on which web browser functionally is provided. Thus, access to the surgical media may be efficiently and economically provided to users of the platform. Furthermore, the surgical media data management platform provides secure communication and storage of data from the source of the surgical media data through to delivery of the media data to users of the platform. This includes both transmission to the platform as well as any distribution including viewing of archived data or live-streams of the data. In turn, the platform complies with regulatory requirements for the protection of patient data through encrypted communications, user access management, activity logging, and other security features described herein.

Accordingly, with proper management of videos, training can be provided, enhanced care may be facilitated, and review of critical content with legal teams (such as to prove that during an untoward event, the surgical decisions were precise to the standard of care) may be provided. Further, proper management of such content can curtail and avoid potential governmental privacy-related fines for mismanagement of the data. As a perfect record of the surgical case, the video also thus can be a performance improvement tool-surgeons, staff, administrators and colleagues can learn from a surgical video infinite wisdom that relates to the patient outcome and the patient experience overall.

In one aspect of the present disclosure, a surgical media management system is provided. The system includes a network-connected source computing device operative to receive surgical media data. A source real-time communication (RTC) client executing on the client computing device is configured for encrypted communication of the surgical media data from the client computing device in real-time. In turn, a cloud-hosted surgical media management platform is operative to receive the encrypted surgical media data communicated from the source RTC client of the source computing device at least for storage of the surgical media data in a cloud storage instance. A media access portal of the surgical media management platform is accessible via network communications to authenticate remote access to the surgical media data in the cloud-hosted surgical media management platform.

This Summary is provided to introduce a selection of concepts in a simplified form that is further described below in the Detailed Description. This Summary is not intended to identify key features or essential features of the claimed subject matter, nor is it intended to be used to limit the scope of the claimed subject matter.

Other implementations are also described and recited herein.

### BRIEF DESCRIPTIONS OF THE DRAWINGS

FIG. 1 is a schematic view of an example surgical media management system.

FIG. 2 is a schematic view of an example surgical media management system with peer-to-peer media exchange mediated by a surgical media management platform.

FIG. 3 is a schematic view of an example surgical media management system with a detailed schematic of an example source real-time communication client.

FIG. 4 is a schematic view of an example surgical media management system with a detailed schematic of an example surgical media management platform.

FIGS. 5-17 are screenshots of various interfaces of an example surgical media management platform.

FIG. 18 is a schematic view of an example, processing system capable of executing aspects disclosed herein.

### DETAILED DESCRIPTIONS

The following description is not intended to limit the claimed invention to the forms disclosed herein. Consequently, variations and modifications commensurate with the following teachings, skill and knowledge of the relevant art, are within the scope of the present invention. The examples described herein are further intended to explain known modes of practicing the claimed invention and to enable others skilled in the art to utilize the claimed invention in such, or other, examples and with various modifications required by the particular applications(s) or use(s) of the presently claimed invention.

As used in the claims and in the corresponding portion of the specification, the word “a” means “at least one”. Further, unless otherwise defined the word “about” when used in conjunction with a numerical value means a range of values corresponding to the numerical value plus or minus ten percent of the numerical value. Still further, the word “or” has the meaning of a Boolean inclusive “Or”. For example, the phrase “A or B” means “A” alone or “B” alone or both “A” and “B”.

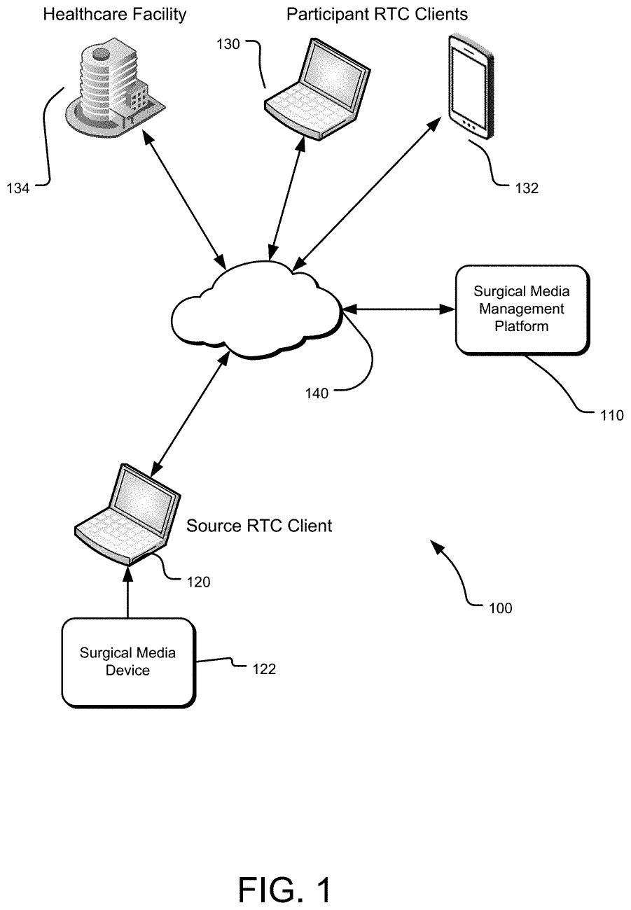

FIG. 1 depicts an example of a surgical media management system 100 according to the present disclosure. The surgical media management system 100 generally includes a surgical media management platform 110. A source real-time communication (RTC) client 120 is executed on a source computing device and is in operative electronic communication with the surgical media management platform 110 by way of network 140. As will be discussed in greater detail below, the source RTC client 120 is used to provide surgical media data from a surgical media device 122 to the surgical media management platform 110. In turn, the surgical media management platform 110 stores the surgical media data in a cloud-based storage structure. As described above, communication of the surgical media data from the source RTC client 120 to the surgical media management platform 110 may be via secure communications over network 140. For example, the surgical media data may be encrypted (e.g., via transport layer security (TLS) using an appropriately robust cryptographic protocol).

In addition to providing storage of the surgical media data at the surgical media management platform 110, the surgical media management platform 110 may facilitate a secure live-stream broadcast of the surgical media data to one or more participant RTC clients executing on one or more corresponding participant computing devices. For example, participant RTC client 130 and participant RTC client 132 are shown in FIG. 1. However, additional or fewer participant RTC clients may be provided without limitation. The source RTC client 120 may establish a peer-to-peer data connection with the participant RTC client 130 and/or participant RTC client 132 that is mediated by the surgical media management platform 110 for purposes of the live-streaming or real-time broadcast of the surgical media data to the participant RTC client 130 and/or participant RTC client 132. The participant RTC clients 132 may be at any appropriate location including those remote from the surgical theater. This may be particularly useful in the context of sharing data with medical device providers or in cases where the surgical theater is in a dangerous location such as a battlefield hospital or the like.

Moreover, a healthcare facility 134 (e.g., hospital, outpatient clinic, etc.) may be in operative electronic communication with the surgical media management platform 110 via network 140. The healthcare facility 134 may have a participant RTC client and/or may access the surgical media management platform 110 to receive live-stream or archived surgical media data. In addition, the healthcare facility 134 may provide healthcare data to the surgical media management platform 110 via a healthcare informatics interface, which may be associated with appropriate surgical media data as discussed further below. In turn, the surgical media management platform 110 may apply machine learning or other artificial intelligence to the surgical media data and/or the healthcare data provided by the healthcare facility 134 to provide insights or other data analytics regarding the surgical media data. Such data analytics (e.g., AI techniques) may be performed in real-time as the data is being broadcast or may be applied to surgical media data stored in a library. In this regard, the surgical media data may be stored in the cloud storage instance of the surgical media management platform 110 in a structured media library in which surgical media data and/or metadata regarding the surgical media data are stored in an structured and/or indexed library.

The network 140 may be a wide area network (WAN) such as the Internet. In other examples, any appropriate communication network 140 may be utilized to provide communication with the surgical media management platform 110. Such networks may include local area networks (LANs), wireless networks, cellular data networks, publicly switched telephone networks, etc. Additionally, while shown as a single instance in FIG. 1, the surgical media management platform 110 may actually comprise a plurality of nodes in a cloud-based architecture. For instance, the surgical media management platform 110 may include a plurality of cloud storage instances or containers to store the surgical media data received. Moreover, the surgical media management platform 110 may include single-tenant architecture to provide heightened security to a healthcare facility 134 or other entity employing the surgical media management platform 110. In other approaches, a multi-tenant environment may be provided in which access to surgical media is stored in a common data repository with limits on access to the surgical media data via user access and authentication controls. In this regard, the surgical media management platform 110 may be resident in any one or more standard cloud-based computing service including Amazon® Web Services (AWS®), Microsoft® Azure®, Google® Cloud Platform, Oracle® Cloud Infrastructure, or another appropriate cloud-based computing service.

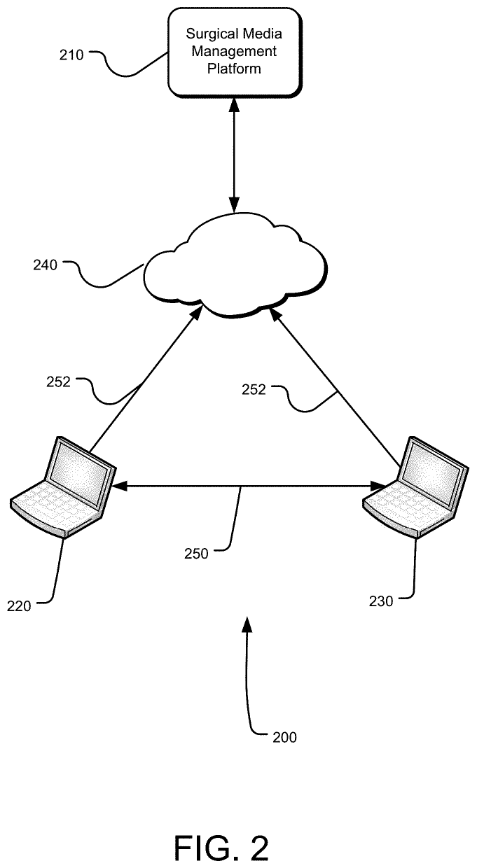

As noted above, the surgical media management system 100 may facilitate a live stream of surgical media data from a source RTC client 120. Specifically, such a live stream or real-time broadcast may be facilitated using a real-time communication architecture that is mediated by the surgical media management platform 110. With further reference to FIG. 2, one such RTC system 200 is depicted. In FIG. 2, a source RTC client 210 provides a live stream or real-time broadcast of surgical media data 250 to at least one participant RTC client 230. Thus, while only one participant RTC client 230 is depicted in FIG. 2, it may be appreciated that additional participant RTC clients may be provided without limitation. In general, the source RTC client 220 may communicate with a surgical media management platform 210 via network 240. In addition, the participant RTC client 230 may communicate with the surgical media management platform 210 via network 240. The surgical media management platform 210 may include or provide an RTC service through an RTC protocol. In general, RTC protocols leverage application programming interfaces (APIs) to coordinate a peer-to-peer connection between computing nodes to share media data therebetween. Examples of appropriate RTC protocols include the Real-Time Transport Protocol (RTP), Real-Time Streaming Protocol (RTSP), Session Description Protocol (SDP), or other appropriate protocols for providing real-time communication of media data such as video and/or audio. One particular example of an RTC protocol that may be used is the webRTC protocol, which is a standardized protocol provided through the world wide web consortium (W3C) and the Internet Engineering Task Force (IETF).

The surgical media management platform 210 may include or call remotely to an RTC engine that may provide support for the peer-to-peer communication of surgical media data 250 from the source RTC client 220 to the participant RTC client 230. For instance, the surgical media management platform 210 may negotiate session initiation, user authentication, security key management, or other overhead requirements for the peer-to-peer connection. In addition, the surgical media management platform 210 may generate and provide user interface elements to the source RTC client 220 and/or participant RTC client 230 for ancillary communication tools associated with the surgical media data 250 such as media annotation, chat functionality, source selection, or the like, as described in greater detail below.

In any regard, the source RTC client 220 may communicate signaling data associated with the live broadcast of the surgical media data 250 via the network 240 to the surgical media management platform 210. In turn, the surgical media management platform 210 may exchange signaling data 252 with the participant RTC client 230. The signaling data 252 may facilitate the peer-to-peer exchange of the surgical media data 250 between the source RTC client 220, and the participant RTC client 230.

In addition, the source RTC client 220 provides the surgical media data 250 that is communicated to the participant RTC client 230 via a peer-to-peer connection to the surgical media management platform 210 for storage of the surgical media data 250 in the surgical media management platform 210 (e.g., in the cloud storage resources of the surgical media management platform 210).

As may be appreciated, the surgical media data 250 communicated in real-time to the surgical media management platform 210 may be in a format associated with an RTC protocol used by the source RTC client 220. For instance, a specific RTC format for encoding media data (e.g., audio and/or video) for live streaming or broadcast of the data may be utilized. Examples of such formats may include the WebM format, third generation partnership program (3GPP) format, Advanced Systems Format (ASF), DivX Media format, Flash Video format, Matroska Multimedia Container format, or the like.

In this regard, the RTC system 200 may leverage appropriate RTC technology that allows the source RTC client 220 and participant RTC clients 230 to implement the exchange of surgical media data 250 with very low overhead and with standard computational devices (e.g., laptop computers, desktop computers, tablet computers, smartphones, a dedicated surgical media control device, or any other web browser equipped computing device). For example, the source RTC client 220 and/or participant RTC client 230 may comprise a web browser having appropriate API access capacity to negotiate the exchange of signaling data 252 associated with the RTC system 200. While the source RTC client 220 and/or participant RTC client 230 may be a desktop application installed on a user's computing device, such an application may still utilize the web-based API calls to facilitate the exchange of signaling data 252 to support the peer-to-peer exchange of surgical media data 250 in real-time. Moreover, all API communication may be encrypted and/or subject to user authentication, thus facilitating a secure peer-to-peer exchange. By using a web-based infrastructure for the surgical media management platform 210, source RTC client 220, and participant RTC client 230, the need to download, install, and maintain programs, plug-ins, applets, or other software subject to upkeep and security risks may be eliminated. This approach is in contrast to traditional approaches that require costly content delivery networks and/or private IP TV mechanisms or proprietary and often cost-intensive hardware or software (not limited to but including proprietary codecs) and possibly involving dedicated routers and other hardware installed at content delivery network location (i.e., a point of presence). As described in greater detail below, the source RTC client 220 may be a dedicated hardware appliance with functionality limited to the initiation and recording of surgical media data 250 for storage at the surgical media management platform 210. That is, the source computing device may facilitate exclusive execution of the source RTC client 220 such that the cost, complexity, size, and/or operational complexity of the source computing device is reduced or minimized.

The platform of the present disclosure has profoundly lower costs per bit of data moved, stored, and other functionality provided with the exchange of surgical media data. This provides the healthcare provider IT team with a qualified, regulatory compliant and IT policy-satisfying data delivery, data management and cloud infrastructure that is welcome according to IT concerns set forth above. The RTC system 200 is also at a relatively inexpensive cost and does not require the IT department of the healthcare provider to manage such functionality locally. With no burden of support, management or other labor effort or expense the overall cost of the RTC system 200 and surgical media management system 100 is reduced. That is, the surgical media management system 100 may be managed by a third party (e.g., a platform provider), yet allows for insights to be provided to the healthcare provider's IT department regarding all of the data delivery, data management, and cloud infrastructure to allow for the healthcare provider to meet regulatory and other IT policy concerns such as audits, response to HIPAA violations, breaches, etc. In addition, surgical media storage can be maintained on unique storage repositories such that the ownership (since managed logically and not physically such as on-premise devices) can be held by the healthcare provider. In turn, keys (e.g., cryptographic keys or the like) to the storage repositories are held by the healthcare provider and not by an outside party. Moreover, the surgical media management platform 210 may coordinate cloud storage into a healthcare facility's own cloud infrastructure for more robust management by the facility of the data.

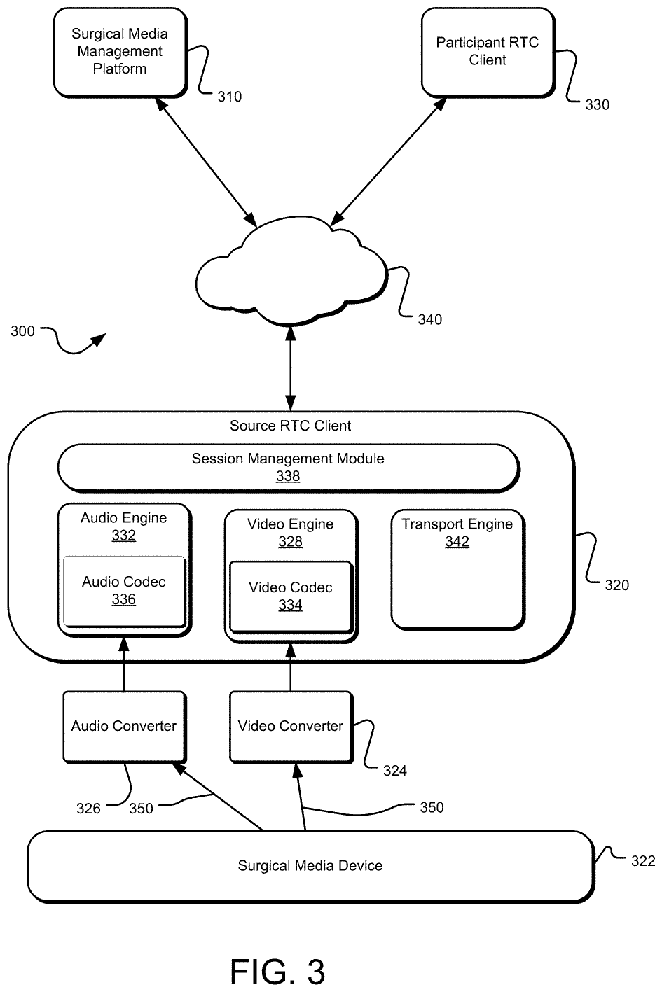

With further reference to FIG. 3, a surgical media management system 300 is shown with a detailed example of a source RTC client 320 depicted. As generally described above, the source RTC client 320 may receive surgical media data 350 from a surgical media device 322. The source RTC client 320 may, in turn, provide the surgical media data 350 to a surgical media management platform 310 and/or participant RTC client 330 via network 340.

However, the surgical media device 322 may output surgical media data 350 in a format that is not recognized or otherwise useful to the source RTC client 320. In turn, a video converter 324 may receive surgical media data 350 from the surgical media device 322 in the native video format output by the surgical media device 322. In turn, the video converter 324 may convert the surgical media data 350 to an input video format recognized or otherwise readable by the source RTC client 320. The video converter 324 may be an external device to a source computing device on which the source RTC client 320 executes or may be integrated with a source computing device on which the source RTC client 320 executes. An audio converter 326 may receive surgical media data 350 from the surgical media device 322 in the native audio format output by the surgical media device 322. In turn, the audio converter 326 may convert the surgical media data 350 to an input audio format recognized or otherwise readable by the source RTC client 320. The audio converter 326 may be an external device to a source computing device on which the source RTC client 320 executes or may be integrated with a source computing device on which the source RTC client 320 executes. In addition, the video converter 324 and the audio converter 326 may be provided as an integrated unit rather than separate modules, as shown in FIG. 3.

The source RTC client 320 may include a video engine 328 and an audio engine 332. The video engine may include video processing capabilities to ready the surgical media data 350 for distribution to the surgical media management platform 310 and/or the participant RTC client 330. In this regard, a video codec 334 may be provided to reformat the surgical media data 350 from the input format into a real-time format as described above. The video engine 328 may also provide image enhancement such as noise reduction or dynamic jitter buffering to enhance the quality of the surgical media data 350 to be provided from the source RTC client 320. Also, an audio engine 332 may be provided. The audio engine 332 may include an audio codec 336 to reformat the surgical media data 350 from an input format into an audio format suitable for real-time communication. The audio engine 332 may also include an echo canceller and/or noise reduction to enhance the audio portion of the surgical media data 350 if any. The audio engine 332 also may connect to a speech-to-text function, which can convert recorded audio content to text, which can later be used for searchability, training, analysis and inclusion into artificial intelligence processes. Again, while the video engine 328 and audio engine 332 are shown as separate modules in the source RTC client 320, the video engine 328 and the audio engine 332 may be integrated into a single module with one or more of the video codec 334 and/or audio engine 332. In any regard, upon processing by the video codec 334 of the video engine 328 and/or the audio codec 336 of the audio engine 332, the source RTC client 320 may communicate the surgical media data 350 to the surgical media management platform 310 and/or participant RTC client 330 in the real-time transport format associated with the real-time protocol utilized by the surgical media management system 300.

The surgical media device 322 may be any appropriate medical imaging device including, potentially, media (e.g., video, 3D video, and/or audio) input from cameras, scopes, surgical robots, imaging devices, radiological information capture or the like. The output surgical media data 350 may be provided from a video cable from the surgical media device 322. The surgical media device 322 can be but is not limited to, a surgical camera often via an SDI (serial digital interface) or DVI (digital video interface), HDMI (High-Definition Multimedia Interface) or analog video connection on the surgical camera “stack” or computing device, or the SDI, DVI or HDMI output of a surgical video source on a surgical robot or other surgical system that involves the capture of video. The video converter 324 converts the signal from a source format to a desired input format for the computing device executing the source RTC client 320. In some embodiments, the video converter 324 and/or source RTC client 320 may provide conversion of the video signal to fiber optic (to meet particular biomedical engineering concerns at some locations globally). In addition, possible conversion of the video signal may be provided over a secure wireless video system. In turn, the video converter 324 may facilitate the conversion of the fiber optic video to the desired format for the computing device of the source RTC client 320. The video converter 324 may include a “video frame grabber,” for injection of the video signal is to the source RTC client 320.

The source RTC client 320 also includes a transport engine 342. The transport engine 342 may include an RTC protocol stack and/or components that facilitate session generation and maintenance between the source RTC client 320 and the surgical media management platform 310 and/or participant RTC client 330 across various types of networks. The source RTC client 320 may also include a session management module 338. The session management module 338 is an abstracted session layer allowing for call setup and management of sessions between the source RTC client 320 and the surgical media management platform 310 and/or participant RTC client 330. For example, the session management module 338 may have API calls to facilitate aspects of the RTC peer-to-peer session between the source RTC client 320 and the surgical media management platform 310 and/or between the source RTC client 320 and the participant RTC client 330.

As will be described in greater detail below, the source RTC client 320 and/or participant RTC client 330 may be executed by respective processing system (e.g., a laptop computer, tablet computer, or the like). The source RTC client 320 and participant RTC client 330 are specifically architected to reduce the technical requirements of the processing system executing the source RTC client 320 or participant RTC client 330. For example, commodity and often easily purchasable parts or systems may be used to execute the source RTC client 320 and/or participant RTC client 330. The small hardware components that may be used, such as the video converter 324 and/or audio converter 326, are also designed to be inexpensive and straightforward.

In at least one example of the surgical media management system 300, the hardware components (e.g., the processing system executing the source RTC client 320) may meet the “medical grade standard” of the International Electrotechnical Commission (IEC) 60601 specification. Such requirements may be needed in some applications of operating theatre technologies, such as being placed near the “patient vicinity.” Although in other contexts, the hardware components do not need to be placed near the patient vicinity, the product may be qualified as a medical-grade IEC 60601 technology. In addition, the video converter 324 and/or audio converter 326 may all be certified for IEC 60601 compliance in order for the perioperative team to have a safe technology. Note that a medical grade IEC 60601 personal computer is often used, however, because many hospitals deploy personal computers in the operating theatre that are widely commercially available that are not medical grade IEC 60601 certified, a non-medical grade personal computer can be used.

As described above, in an example of the surgical media management system 400, a specific dedicated hardware component may be provided for execution of the source RTC client 320. In turn, the source computing device executing the source RTC client 320 may be a small, portable medical-grade hardware comprising a system on a chip for exclusive execution of the source RTC client 320. Such a source computing device may include appropriate connections to facilitate connections to any video source in the operating theatre. The source computing device may also be used without a network connection or with a wired or wireless network connection. In one example, the source computing device may include a power over ethernet power source. Such a dedicated hardware component may include an interface for receipt and display of a user interface associated with execution of the source RTC client 320.

In this example of a source computing device, the device may include a simplified user experience that allows a user to log in and to initiate recordings of surgeries, either as a new session with new metadata or by selecting an available session on the product menu of sessions populated from information such as from a surgical scheduling system, patient admissions system, or the surgical media management platform 410. The recorded video can be previewed, edited, and metadata updated. In turn, the video may be uploaded at the moment of capture or on a user-defined schedule. In turn, after upload, the video may be available via the media access portal 426 of the surgical media management platform 410.

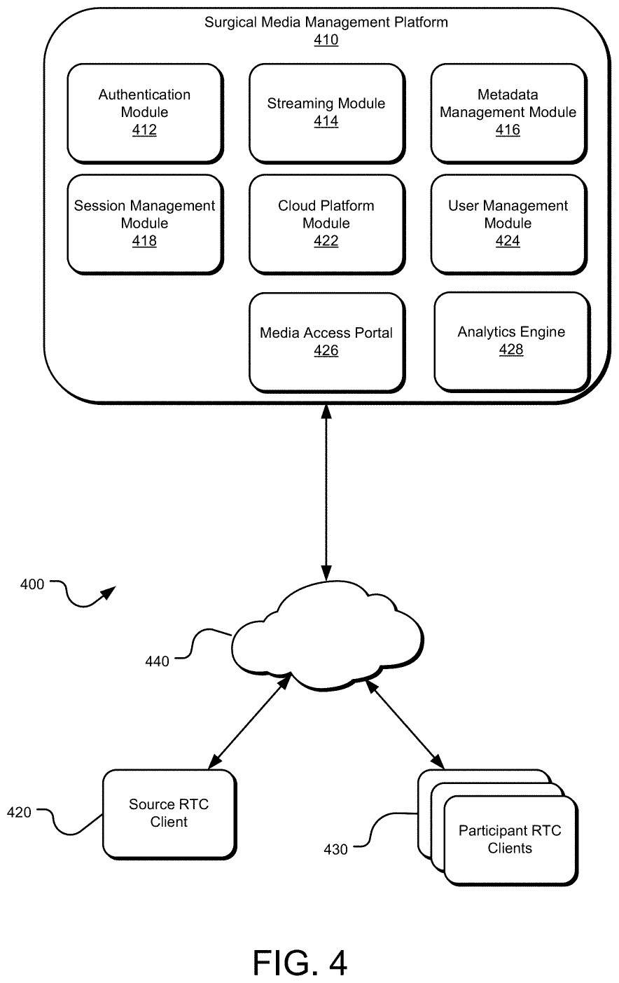

With further reference to FIG. 4, a surgical media management system 400 is depicted in which an example surgical media management platform 410 is shown in a detailed view. As described above, a source RTC client 420 may provide surgical media data 450 to the surgical media management platform 410 and/or participant RTC client 430 via a network 440.

The surgical media management platform 410 includes a media access portal 426 that may provide a user interface for interaction with the surgical media management platform 410. For instance, the media access portal 426 may provide a user interface to the source RTC client 420 and/or the participant RTC client 430 in connection with the peer-to-peer media exchange between the source RTC client 420 and the participant RTC client 430. The media access portal 426 may also provide access to a source RTC client 420 and/or participant RTC client 430 for access to archived surgical media data 450 stored by the surgical media management platform 410 as described in greater detail below. In an example, the media access portal 426 facilitates access to the structured media library of surgical media stored at the surgical media management platform 410. This may allow a user or analysis tool (e.g., analytics engine 428 described below) to search the structured surgical media library stored in the surgical media management platform 410. The analysis tool may be leveraged to gain insights for a number of stakeholders including healthcare facilities, healthcare providers, medical device companies, governments, and/or militaries.

The surgical media management platform 410 also includes an authentication module 412. A user may be associated with an account on the surgical media management platform 410. In turn, the user may access the surgical media management platform 410 (e.g., by way of a browser navigated to a uniform resource locator for the surgical media management platform 410). The authentication module 412 authenticates the user. The authentication by the authentication module 412 may involve both username and password combination and a two-factor authentication whereby a code is used to authenticate a user. The authentication module 412 also captures the public internet protocol (IP) address of the user at the login attempt for security and tracking purposes. The authentication module 412 can also integrate with a single sign-on (SSO) and/or Active Directory.

A streaming module 414 is provided in the surgical media management platform 410. The streaming module 414 may facilitate aspects of real-time communication of surgical media data 450 between a source RTC client 420 and one or more participant RTC clients 430. The streaming module 414 may include an RTC engine that provides signaling data for one or more peer-to-peer connections for the exchange of surgical media data 450.

The surgical media management platform 410 also includes a metadata management module 416. The metadata management module 416 may manage metadata associated with surgical media data 450, as described in greater detail below. The metadata management module 416 may also receive metadata from external sources (e.g., a health informatics interface of hospitals or healthcare providers including an HL7 feed or other appropriate HIS system feed). Inclusion of such metadata may enhance or facilitate artificial intelligence or machine learning as will be described in greater detail below. Such integration with a health informatics system may be used to supplement metadata for surgical media data once stored or may be used during the recording of the surgical media data to pre-populate metadata fields for the session. In this regard, the metadata management module 416 may comprise any appropriate health informatics interface to receive, parse, and/or interpret healthcare data received at the surgical media management platform 410. The metadata management module 416 may also maintain and/or manage the structured media library including through indexing or other library services.

As described above, the surgical media management platform 410 may be hosted in a cloud-based architecture meeting conventional IT concerns (such as compliance with Statement on Auditing Standards No. 70 (SAS-70)), behind load balancers that block and limit traffic for security and vulnerability concerns (such as limiting all access to the customer infrastructure to port 443). In this regard, port 443 is used with a 256-bit encryption or any level encryption required in order to meet conventional healthcare IT security concerns and Health Insurance Portability and Accountability Act (HIPPA) rules in the U.S. The surgical media management platform 410 may be hosted for a given healthcare facility or provider on its own single-tenant infrastructure (though in certain instances, surgical media data 450 may also be on a carefully architected multi-tenant infrastructure) for data privacy and other conventional healthcare IT department concerns.

The surgical media management platform 410 also includes a user management module 424 for the management of authorized users of the surgical media management platform 410. User accounts are set up into groups. These groups may have different privileges. For example, “administrators” may invite guests to create accounts, create session rooms, invite guests to view recorded videos, control permissions on sessions, videos, and/or users, and/or start and stop recordings in a session. Users may alternatively be “publishers” may have various lower level of controls, such as the ability to stop and start recordings in a session. “Members” may have an even lower level of access, which allows users in the members' group to access sessions and videos. “Guests” (who are likely not associated with a hospital or healthcare provider) have less permission and need all session and/or video access delegated to them by an Administrator. Such permissions may be established via the user management module 424, which may be accessed by the media access portal 426.

The user management module 424 may allow users with appropriate permissions to add, modify, and/or delete sessions, invite users to sessions, invite new users to create an account via their email address, add, modify, and/or delete videos or recordings (including the video metadata), modify other metadata used by the surgical media management platform 410, access statistics about usage of the surgical media management platform 410. The statistics of the surgical media management platform 410 may include login failures, login successes, a record of emails sent by the surgical media management platform 410, what videos were accessed by what user at what date/time, if a two-factor authentication code was resent to a user, when a user accessed any particular session, and/or when a forgot-password email was sent to a particular user. All data in the statistics may include IP address information captured for each user. A user may be able to search the statistics page. Statistics logging and reporting can be customized to meet local healthcare information management and reporting requirements. Additional logs regarding the performance of the platform 410 may also be provided including, without limitation, activities related to security auditing, billing, or the like.

The user management module 424 may allow individual users to modify their account profile information and/or delete their account

The surgical media management platform 410 may include a system test page to check if the user's computer and/or network function appropriately for the surgical media management platform 410. On the surgical media management platform 410, a user can also access a network check page to gauge their upload, download, jitter, and/or delay aspects for review.

The surgical media management platform 410 also includes a session management module 418, which may manage access to sessions and/or features provided in the session. For example, an authenticated user (i.e., a user authenticated by the authentication module 412) can join a session if appropriate permissions are provided to the user. As will be described in greater detail below, access to a session may be by way of a user interface (e.g., a web-based user interface) provided by the media access portal 426. The media access portal 426 may have an access page, where thumbnails to permitted sessions for the user are available for selection. Upon selection of a session, a user with permissions that require authorization may request access by an administrator for the user's admission into the session for approval by the administrator. This may assist in reducing the likelihood that a guest views content or patient information that they are not authorized to view.

Upon admission of a user to a session, the session management module 418 may provide a notification to existing users in a session in real-time of the newly arrived guest. This may prompt for approval or rejection of the admittance of the user to the session. The notification may include a display and/or auditory cue indicating a user is requesting to join. The notification preferences may be set by individual user preference.

If approved, the guest joins the session. If rejected, the guest is brought back to the access page and notified of the rejection and with a message to contact the Administrator or the hosting party if there is a question regarding the rejection.

Once a user is admitted to a session, the user may publish a single video or audio source. The source may be selectable. In this regard, the video source can be an internal webcam, external webcam, or another video source of the user's device. The selection of the source is performed through a menu illustrated below. An audio source may also be selected from, for example, a built-in webcam microphone, another built-in microphone, or other audio device input of the user's device.

The user can also publish a second video or audio source for sharing with the session. In turn, the surgical media data 450 may include multiple streams of audio and/or video sources from a user. Thus, for example, the broadcast of an internal video view (such as an endoscopic camera) and an external view (such as a webcam or other video source) may be simultaneously selected for inclusion in the surgical media data 450 that is received at the surgical media management platform 410 and/or broadcast to a participant RTC client 430. This may further be in addition to a screen share provided by a user. The user can enable, disable, or change the media sources throughout the duration of the session. The user can also share their local screen or a local file in the session using a screen share feature. The user can also share just a specific application running on the user's computing device in a session.

The user interface provided by the media access portal 426 may allow a user to view thumbnail images in a source selection list for other users in the session. Several parties and their multiple media sources may be connected in a session (e.g., simultaneously). The user may select on any source thumbnail to make that source the focus of the user's session in which the source is enlarged. In addition, a user may mute another user to preclude audio and/or video source from being seen by the user who muted the another user.

As will be described in greater detail below, the session management module 418 may allow a user to make annotations relative to one or more sources of surgical media data 450 (e.g., by drawing on the video feed). In turn, this feature may allow for teaching, explanation, or making visual emphasis for parties to see while seeing the video being broadcasted. Drawing may be facilitated using a mouse or touchscreen. Drawings/markings may be made public such that all participant RTC clients 430 and the surgical media management platform 410 receive the annotation or private such that only the user making the annotations may view the annotations. A user may also place and/or move a pointer relative to the surgical media data 450. Different users may modify the location and/or appearance of the pointer.

In addition, the session management module 418 may facilitate a chat feature for users of the session. All users can type in messages and view the text messages of other users. In addition, private chats among select users of the session may also be provided. If the CHAT window is not open and a chat has begun, the user with the window not open is notified of the chat in progress.

Further still, the session management module 418 may provide a help interface that may open on that user's screen. In turn, the user may be able to, from the help interface, start an immediate phone call (using the screen they already have open on the surgical media management platform 410) and talk with a technical support team. Additionally or alternatively, a user may start an immediate phone call with any phone in the world, by merely inputting the phone number and pushing the call button from the help interface. This starts a phone call that all parties of the session can hear. Further still, the help interface may include a pre-defined list of users and/or other personnel with particular medical specialties. Accordingly, when a user clicks on a medical specialty, the user may be presented with the names of surgeon colleagues of that specialty. The user can click on any name to start either a phone call directly with that party (in the application) or send that party a pre-formatted email urging them to meet in the session (e.g., the message provided to the third party may include a direct link to the session for directly joining the session on the surgical media management platform 410).

A user may access a participant interface in which participants of the session are shown. This can present a user's name, their medical specialty, or any other custom metadata. An Administrator can use the participants' interface to remove any party immediately from the session and/or revoke access privileges. The user can click on an exit button to leave the session. A party that exits the session will no longer be audience to the broadcasted surgical media data 450, and will no longer be broadcasting their own media, and will be notified of such.

Users with appropriate permissions may start a recording of the session. A recording indicator is shown to all users of the session when the recording starts. Each user has a recording made of the media stream (audio and video) provided by the user. Any user who arrives after a recording has started becomes part of the recording at the time they enter the session. A user with appropriate authority can stop a recording at any time. When the recording is stopped, the recording indicator disappears, and users of the session may be notified. A recording can be started again at any time in the session.

A recording can be tagged with metadata inputted by the user or associated with the session (and confirmed by the user). Such metadata tagging may include integration over a hospital information system (HIS) to retrieve and automatically associate metadata with the surgical media data 450. This may include including tagging of individual or multiple currently procedural terminology (CPT) or ICD-10 codes, which may be stored in the surgical media management platform 410.

The surgical media management platform 410 also includes a cloud platform module 422. The cloud platform module 422 may manage the archival of surgical media data 450 from sessions hosted by the surgical media management platform 410. As described above, when the surgical media data 450 is received from the source RTC client 420, the surgical media data 450 may be in a real-time transport format associated with the real-time protocol used to exchange surgical media data 450 in a peer-to-peer fashion in real-time. As may be appreciated, such a real-time transport format may be less advantageous than other container formats designed for robust media playback and archiving. As such, the cloud platform module 422, in addition to management of cloud storage instances for storage of the surgical media data 450 may include one or more platform codecs for conversion of the surgical media data 450 from the transport formats to an appropriate container format (e.g., MP4) for storage and archival of the surgical media data 450 in a cloud storage instance. In this regard, if a user seeks to access a historical video from the cloud storage instance, the cloud platform module 422 may retrieve the container format version of the surgical media data 450 from cloud storage for payback to the user via the media access portal 426. The cloud platform module 422 may convert the surgical media data 450 at the conclusion of a session. Prior to completion of the conversion, a user attempting to access the surgical media data 450 may be provided a message indicating the surgical media data 450 is being converted. The surgical media data 450 may be provided in the transport format, which may limit features or quality of the surgical media data 450 prior to the conversion to the container format by the cloud platform module 422.

In this regard, the cloud platform module 422 may allow a user appropriately authenticated by the authentication module 412 to view recorded surgical media data 450 from a session. The surgical media data 450 may be available on a video interface in which a user may see video recordings that their user account has been granted access to view. The user may also search by text across the video library, filter videos of the library by custom metadata including but not limited to date of the session, surgeon name, recency of videos, CPT code for the video, and/or the session name. When a user selects a recording on the video page, they can view the individual recording files of all surgical media data 450 from users that were broadcast in the session. This may include synched surgical media data 450 sources from the users of the session or allow a user to view individual ones of the surgical media data 450 sources from the session individually. As sources are added (e.g., by an existing user adding an additional source or by a new user joining the session), the surgical media data 450 for a given source are shown in this synched mode when the sources became available in the session. The synched playback of the surgical media data 450 may include a notification of when users left the session and/or when sources were added, changed, or removed. An individual user's media can be selected and enlarged during the playback, and any user's audio can be muted during the playback. The user can also click a share button and notify other users about a given video and/or session. The share button may provide a video link to the session's surgical media data and/or the video from a given source of the session. The user can also click a discussion button to add comments or questions to dialog about a video and/or session. This may be useful, especially for educating at academic teaching hospitals. The dialog feature enables various colleagues and students to learn about a video or session, discuss details about that video or session, ask and get answers to questions about that video or session as email notifications are triggered based on the dialog there to the relevant parties. For each video, a user can start a dialog by commenting on the video or posting a question about the video. Users related to the video or on a list to be contacted about that video can all receive email notification of the dialog as new posts are added. The dialog can grow as a stream of learning and training, built by the community of surgeons and other users who are a part of the surgical media management platform 410.

The data flow management by the cloud platform module 422 includes traffic management to ensure that only encrypted connections are allowed and with a transport layer security (TLS) level that meets appropriate specifications (e.g., such as HIPAA in the U.S.).

In some examples, the surgical media data 450 may include three-dimensional videos that allow a user to view the three-dimensional video via a virtual reality headset or another appropriate viewer.

The example surgical media management platform 410 may also include an analytics engine 428. The analytics engine 428 may interface with the metadata management module 416 to receive healthcare data (e.g., from a hospital information system or other electronic medical records source) associated with videos stored by the cloud platform module 422. Specifically, the analytics engine 428 may apply artificial intelligence through machine learning models to identify analytics information regarding the exchange of surgical media data captured in the example surgical media management platform 410.

For example, in order to provide a healthcare provider with the performance improvement, quality enhancement and other data information derived from the use of the invention, media must be captured, provided and stored in the example surgical media management platform 410, and then intelligence applied onto the data using the analytics engine 428.

Such analytics provides a caregiver, a global healthcare institution, or other entities having interest and access the ability to understand, manage and improve the quality of care delivered by analysis of the surgical media data, which may directly enhance surgeon performance, revolutionize surgeon training, lower costs, ensure higher levels of financial reimbursement to care providers, and overall enhance the level of patient care delivered to patients.

The surgical media data 450, once captured and placed into a structured library of the example surgical media management platform 410, is made simple to view and learn from and among a community of learners or surgeons and other healthcare providers. In this regard, the structured library may be provided according to a data model for the surgical media data in the platform. The analytics engine 428 incorporates machine learning to build models, look at patterns, and understand the behaviors of surgeons, as evident in the surgical media data. The result of this is information that a healthcare provider can act on with respect to the data they need for the best care and for best patient outcomes. For example, the metadata management module 416 may integrate with a healthcare data source to associate with the surgical media data recorded by the example surgical media management platform 410 associated healthcare information such as, for example, readmission rates, surgical site infection rates, gathered patient experience survey data, and/or other data points decided upon by the hospital.

The example surgical media management platform 410 may reconcile such healthcare data against information already collected in the example surgical media management platform 410 in the video library and its associated metadata, such as surgeon name, case date, CPT code, International Statistical Classification of Diseases and Related Health Problems (ICD10) code, devices used, duration of cases, duration of specific subsets of activity within the video, or other relevant metadata regarding the surgical media data 450. This metadata may include idle time demarcated in a surgical video document based on an algorithm of the surgical media management platform 410 to detect medical device idle time and translate that into valuable data. Additional metadata information associated with the surgical media data 450 may include OR staff participating in the surgery.

In turn, the machine learning approaches executed by the analytics engine 428 may determine patterns of successful and/or unsuccessful outcomes to the surgical media data metadata by comparing surgical media data to compare metadata between surgeons in the hospital, with cases of the same CPT code, with cases of same or similar ICD10 coding, with cases performed in the same operating theatre number, with cases performed with the same perioperative team, with cases performed with different perioperative teams, with cases performed at the same time of day, or a different time of day, with cases performed on patients with a similar body mass index (BMI), with cases performed on patients with similar anatomical abnormalities, with cases performed on patients with similar (or different) co-morbidities, or by comparing other standard metadata to observe patterns that relate to outcomes. Further, the analytics engine 428 may determine reimbursements of other cases associated with surgical media management platform 410 content of similar CPT or ICD10 coding, with different surgeons, with different case durations, etc. Other metadata regarding the surgical media data may include, without limitation, length of stay, hospital acquired conditions (such as infections), patient experience surveys, Hospital Consumer Assessment of Healthcare Providers and Systems (HCAHPS) scores (which includes 64 metrics in 7 categories), mortality rate, bed utilization, hospital incidents, CMS program performance, average cost per discharge, operating margin, and/or bad debt. The analytics may also be based on recognition or codifying of how devices are used across surgical videos, or other surgeon choices understood through an analysis of the video track, and at times in tandem with audio recorded on the media and processing of that audio with the assistance of speech to text technology.

For instance, the analytics engine 428 may review and compare temporal measurements (e.g., durations) between surgeons and compare that data against reported patient outcomes and readmission rates (or other hospital data) to best understand the merits of one surgeon's performance against another and to refine training and goals at the hospital around such information. In this regard, the metadata management module 416 may include APIs to flexibly and intelligently interface with other information systems. The end result is a set of customized and actionable data, derived from the structured media library acquired by the surgical media management platform 410 and with modeling and intelligence performed by the analytics engine 428, which may include APIs, proprietary code and innovative algorithms of the analytics engine 428.

In turn, the surgical media management platform 410 builds profiles based on the data understood from the now structured media library. That gathered data is then gathered and shaped with a modeling effort. The surgical media management platform 410 then can tune parameters and even help make predictions based on data, patterns and behaviors learned and understood, especially when reconciled against the patient outcome, patient experience and other reported data readily available at the hospital. The surgical media management platform 410 may also enable users to further identify critical points during a case with notes (as described in greater detail below). In turn, the community of users of the surgical media management platform 410 can observe this information and further comment on it. As such, this social interaction data further becomes actionable data (e.g., metadata) the surgical media management platform 410 can use in statistical modeling to provide more data to the hospital regarding performance correlated with the patient outcome, re-admission and surgical site infection data. Therefore, as the surgical media management platform 410 is more intensely used, the surgical media management platform 410 may tag videos automatically based on learned details. This then can lead the hospital to determine when an event, occurrence, or other combination of factors occurs, what actions or other approaches minimize any adverse outcomes, or underscore surgeon actions or choices deemed successful and then disseminate that to surgeon colleagues, perioperative staff or students/fellows/residents to teach others.

The structured data regarding the surgical media data may be intrinsically derived characteristics from the media itself or may comprise extrinsically associated metadata. The intrinsic derived characteristics may include analysis of the media itself to extract the characteristics including, without limitation, analysis of pixel activity, media length, color analysis, machine learning applied to the data captured, or other intrinsically derived data. The externally derived metadata may be provided by a user (e.g., a session participant including the surgeon) or may be associated from a health informatics interface as described above.

The artificial intelligence (AI) approaches applied by the analysis engine 428 may include identification of positive or negative conditions. In this regard, in the case of a positive condition (e.g., a particularly favorable patient outcome), the analysis engine 428 may use AI techniques to identify patterns in the intrinsic or extrinsically derived data to determine what characteristics, practices, techniques, or other conditions are associated with the positive condition. In turn, such conditions associated with the positive outcome may be promoted and/or more widely adopted to help promote the positive condition identified.

Additionally, in the case of a negative condition (e.g., a particularly unfavorable patient outcome), the analysis engine 428 may use AI techniques to identify patterns in the intrinsic or extrinsically derived data to determine what characteristics, practices, techniques, or other conditions are associated with the positive condition. In turn, such conditions associated with the negative outcome may be minimized and/or discontinued to help dissuade the occurrence of the negative condition identified.

While examples are provided above in which a healthcare provider leverages the AI capability of the platform, other stakeholders may also utilize the AI capability. For instance, medical device providers, governments, and/or militaries may also utilize the AI capabilities of the platform to attain relevant actionable data from the library of media data and/or metadata related thereto. For instance, the AI capability may derive training data (to correlate against surgeon performance recognized in the videos). Such training data may be at the level of which medical device company personnel trainer trained a user (e.g., the impact or efficacy of training). Other insights may be obtained based on what sales staff helped educate that surgeon on product use, usage over time of the medical company's device and the device's relationship to surgical performance, outcome data in conjunction with medical device company data of when a product was sold, how a device was installed, how the surgical team was trained and, even down to what region they use the device in including a correlation with regional population data or the like.

The AI approaches used by the analytics engine 428 may include any appropriate machine learning approach. This may include supervised or unsupervised machine learning approaches. Furthermore, the AI approaches may include use of neural networks (e.g., recurrent or non-recurrent neural networks), support vector machines (SVMs), k-means clustering, multivariate analysis, or other AI approaches that may be used to identify patterns or other occurrences in the surgical media data.

Accordingly, the analytics engine 428 may derive actionable intelligence from the application of the AI techniques to the structured library of surgical media data. Such actionable intelligence may include recommendations on surgical best practices, evaluation of surgical performance, tracking of facility characteristics, or any other data that impacts the quality of healthcare provided to patients or operational efficiencies of a healthcare provider.

Effectively, the surgical media management platform 410 enables a healthcare system to use the data derived from the surgical media library generated in the surgical media management platform 410 and, along with data from other sources find answers on how the healthcare provider and its surgeons can best perform surgeries, for better patient care, maximized patient outcomes, and through value based payment or other quality-focused systems more successfully in hospitals, thus providing improved operational efficiencies and increased patient outcomes.

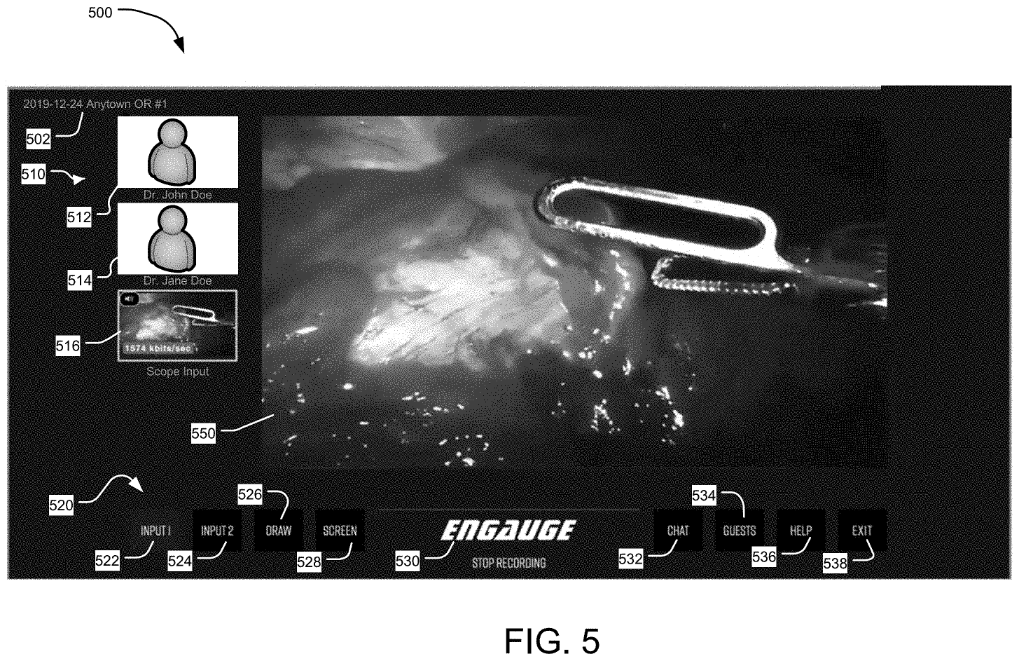

FIGS. 5-17 depict a number of user interface screens that may be used by users during a session and/or during access to a media access portal of a surgical media management platform, as described above. Specifically, FIG. 5 shows a session user interface 500 which may be displayed at a source RTC client. The session user interface 500 may also be presented at a participant RTC client, with the understanding that the participant RTC client may not be in a surgical suite, and thus not have a surgical media input. The session user interface 500 includes a session identifier 502 that provides information regarding the name of the active session.

A media source listing 510 is provided to the left of a main source display 550. The media source listing 510 provides a thumbnail view of each source available in the session. Specifically, in FIG. 5, a first media source 512, a second media source 514, and a third media source 516 are currently active in the session. The first media source 512 may be a web camera that is recording a user (e.g., surgeon Dr. John Doe as shown). The second media source 514 may be a web camera of another user (e.g., surgeon Dr. Jane Doe). Dr. Jane Doe may be observing the surgery performed by Dr. John Doe remotely and may thus have only the web camera interface showing the user. The third media source 516 may be a scope input received from a scope used in the surgery having a video output. That is, the third media source 516 may be from the source RTC client of Dr. John Doe and comprise exchange surgical media data received from the scope used by Dr. John Doe during the surgery. A user (e.g., regardless of whether at a source RTC client or participant RTC client) may click on a selected one of the media sources in the media source listing 510 to provide the selected media source in the main source display 550. For example, in FIG. 5, the third media source 516 is selected by the user for display in the main source display 550. In some instances, a plurality of source RTC clients may be connected to a session such that multiple users are providing surgical media data to a session for distribution and/or storage of the multiple streams at the same time.

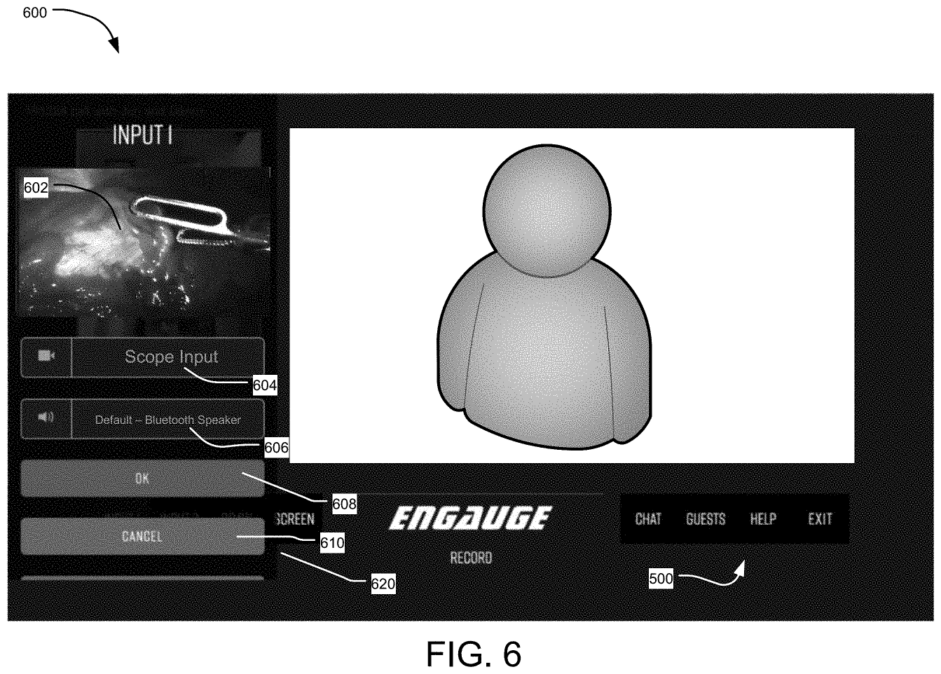

The session user interface 500 also includes a session tool section 520. The session tool section 520 may provide a number of buttons or controls for use in relation to the session. For example, the session tool section 520 includes a first input selection button 522. This may allow a user to select and/or modify a source of media to be provided to the session. Similarly, a second input selection button 524 is provided. In this regard, a user may wish to share at least two sources of media data in the session. For instance, as in the case presented in FIG. 5, the source RTC client of Dr. John Doe may both provide the first media source 512 comprising the web camera input and the third media source 516 comprising the scope input to the display. The first input selection button 522 and the second input selection button 524 are described in greater detail below in FIG. 6.

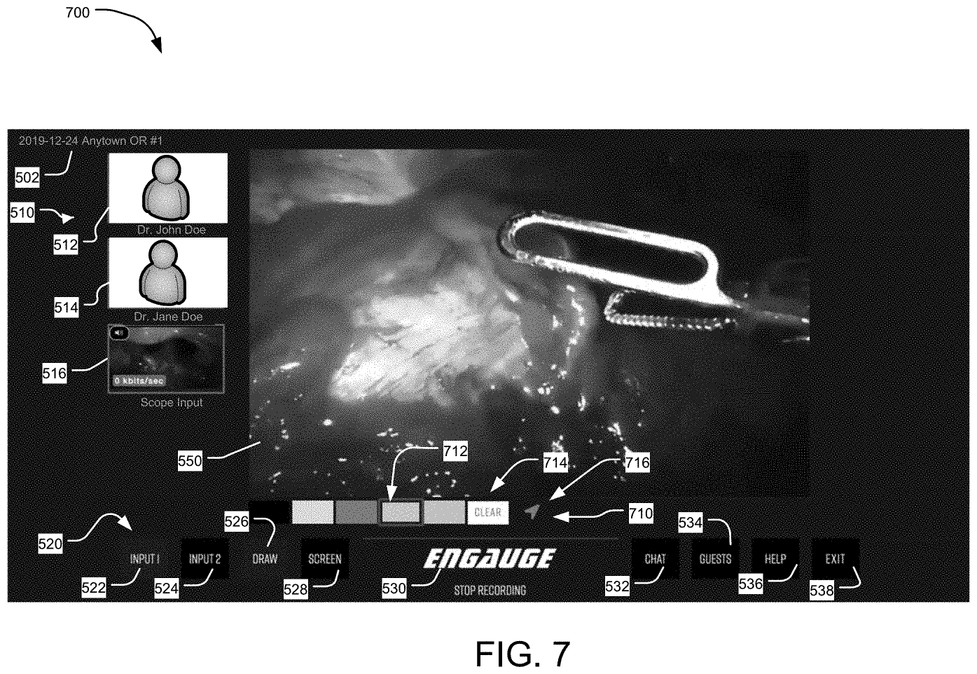

The session tool section 520 also includes a drawing tool button 526. The drawing tool button 526 may allow a user to leverage collaboration tools for explanation, discussion, or other collaboration with other users of the session. Such collaboration tools may include drawing tools for annotation of the main source display 550 during the session as will be described in greater detail below in FIG. 7.

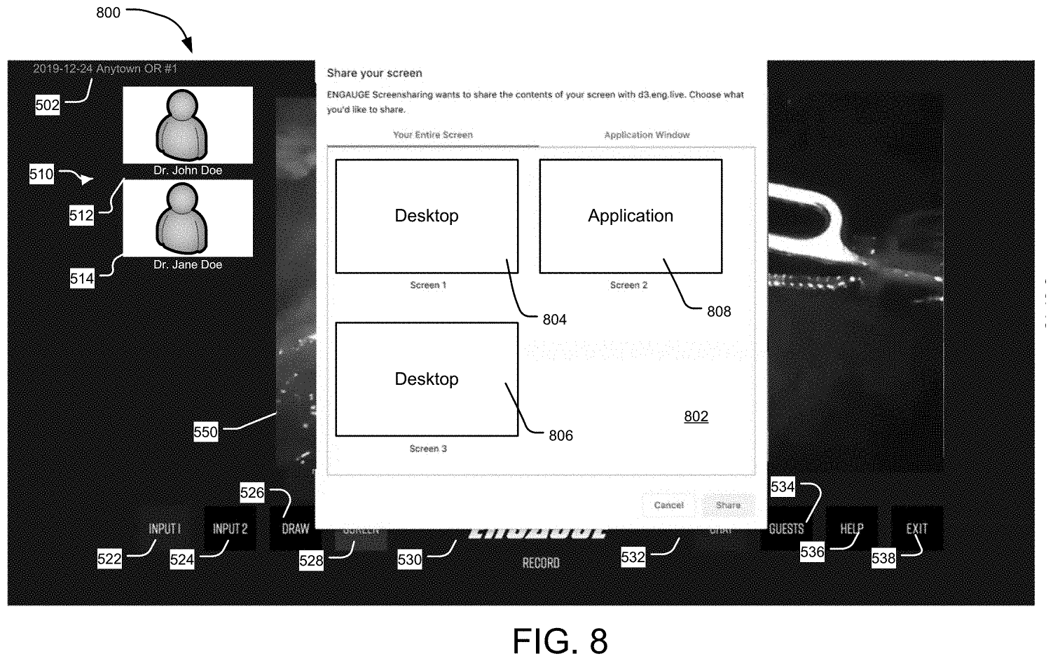

The session tool section 520 also includes a screen share selection button 528 that allows a user to select a screen of the computing device of the user for inclusion in the session. The operation of the screen share selection button 528 is described in greater detail below in FIG. 8.

The session tool section 520 also includes a recording control button 530, which can toggle the recording of the session as described above. The recording control button 530 may be toggled at respective portions of an operation to capture relevant exchange surgical media data. When the recording control button 530 is selected to initiate a recording, a recording indicator (e.g., a highlighted border, user interface message, tone, or the like) may be provided to indicate the session is being recorded. Upon selection of the recording control button 530 to discontinue recording, data regarding the recorded video may be solicited as described in greater detail below. The recording control button 530 may be toggled using an input of the computing device including a mouse clicking the recording control button 530 and/or another means such as a keyboard shortcut or a toggle switch dedicated to the recording control button 530 such as a foot controller or the like. Such a remote controller may be wired or wireless using any appropriate wireless communication technology (e.g., Bluetooth).

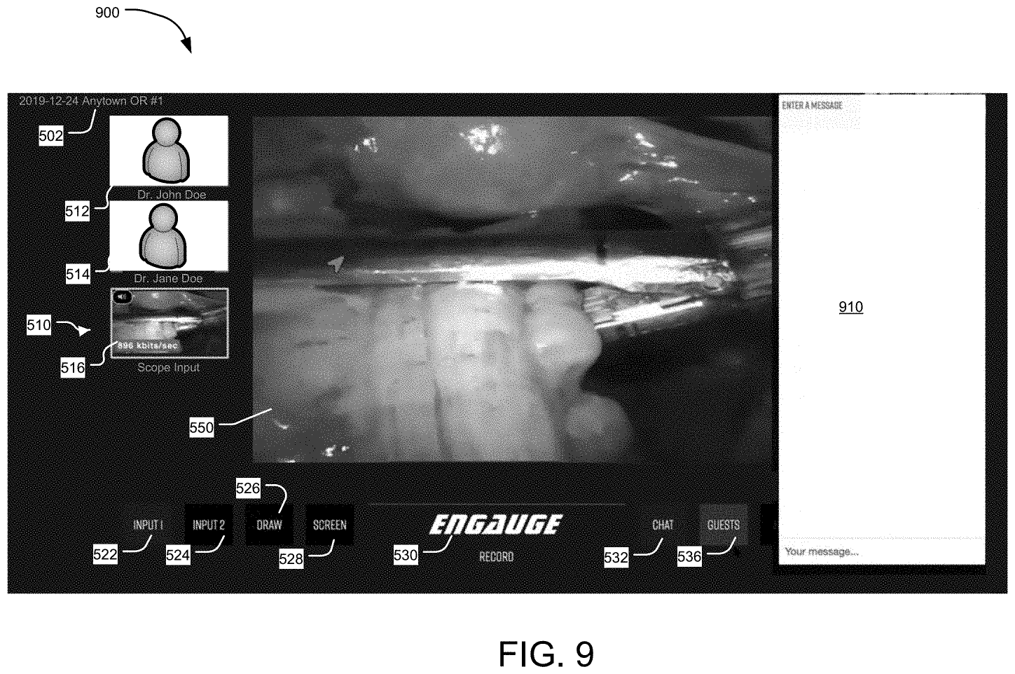

The session user interface 500 also includes a chat button 532 that allows a user to access a chat feature for private or public chats among participants of the session. The chat button 532 will be described in greater detail below in FIG. 9.

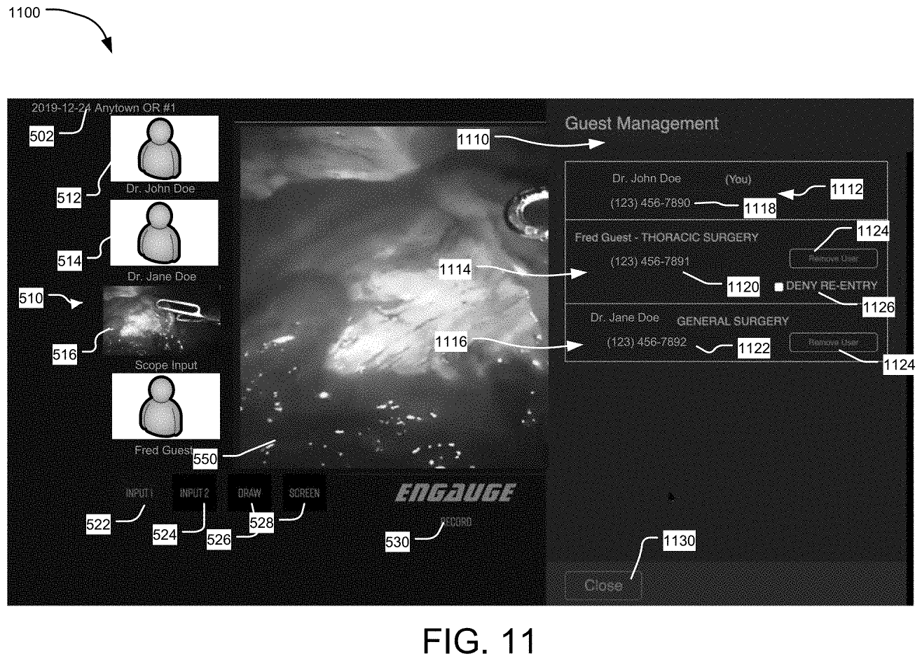

The session user interface 500 also includes a guest management button 534. Selection of the guest management button 534 may allow a user with appropriate permissions to view and/or modify users participating in the session. The operation of the guest management button 534 is described in greater detail below in FIG. 11.

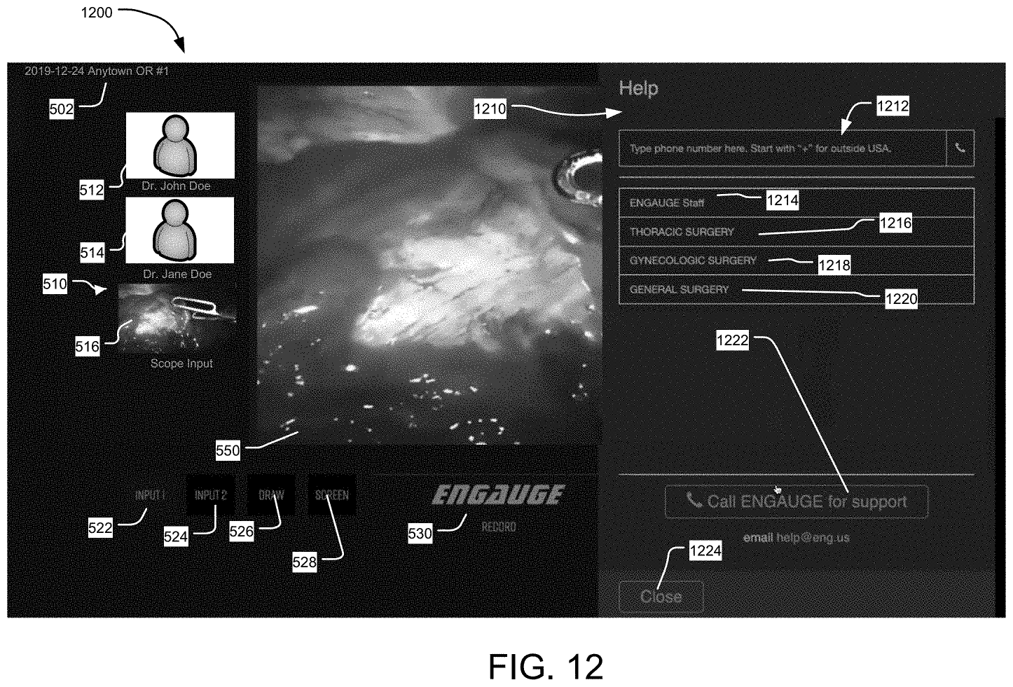

The session user interface 500 also includes a help button 536, which can be used to display a help menu in the session user interface 500. The help button 536 and help menu are described in greater detail below in FIG. 12.

The session user interface 500 also includes a session exit button 538, which allows a user to exit the session.

With further reference to FIG. 6, a source input user interface 600 is shown. The source input user interface 600 may include a source selection menu 620 that is overlaid relative to the session user interface 500 upon selection of the first media source 512 or second media source 514 of FIG. 5. The session tool section 520 may allow a user to select and/or modify a source input to be provided to the session. The source selection menu 620 includes a source preview window 602 in which the selected source may be previewed by the user prior to including the source in the session. A source video selection 604 allows a user to select a video source from among the available video sources of the user's device. This may include, for example, internal web cameras, external web cameras, video cameras, surgical equipment inputs, or the like. The source video selection 604 may include a drop-down menu or another selector to allow a user to select the source. A source audio selection 606 may also be provided for the selection of an available audio source from among those available to the computing device of the user. Once the source selection is complete, a user may select a confirmation button 608 to confirm the selection and return to the session user interface 500 or may select the cancel button 610 to return to the session user interface 500 without saving changes made in the source selection menu 620 A source selection menu 620 may be provided for each of the first media source 512 and second media source 514 such that multiple media sources may be selected for inclusion in the session.

FIG. 7 depicts a drawing user interface 700 that may be provided when a user selects the drawing tool button 526 of the session user interface 500. The drawing user interface 700 includes a drawing toolbar 710 that is displayed relative to the session user interface 500. The drawing toolbar 710 may allow a user to selectively annotate the source displayed in the main source display 550. For instance, a pen color selector 712 is provided that allows a user to select a color for annotations to be provided on the main source display 550 (e.g., through mouse movements, a touchscreen, stylus input, etc.). The drawing toolbar 710 also includes a drawing clearing button 714 that clears all annotations from the main source display 550. A pointer selector 716 may also be provided to allow a user to place a pointer on the main source display 550. Once a pointer is placed, the user or other users may move the pointer relative to the main source display 550 (e.g., to highlight anatomy or other features of interest in the main source display 550). Other tools may be provided in the drawing toolbar 710, including text annotations, stickers, stamps, or other annotations for use with the main source display 550.

FIG. 8 shows a screen share selection interface 800 that may be presented in the session user interface 500 when the user selects the screen share selection button 528. A screen share menu 802 is displayed. In the screen share menu 802, a number of inputs corresponding to displays of the user's computing device are provided. For instance, a user may select a first screen selection 804 or a second screen selection 806 to display respective first or second desktop views of the user's computing device. Upon selection of a screen to share, the video for the selected screen may be included as a media source in the media source listing 510 that is available for selection and recording in the session. Also, the screen share menu 802 includes an application interface selection 808 that may display only a limited portion of a user's screen associated with a given application.

FIG. 9 shows a chat interface 900 that may be presented upon selection of the chat button 532 of the session user interface 500. A chat window 910 may be presented to the user to allow a chat to be initiated between participants of the session. A chat window 910 may be provided as a public chat (i.e., a chat available to all users of the session) or a private chat among a more limited subset of users of the session.

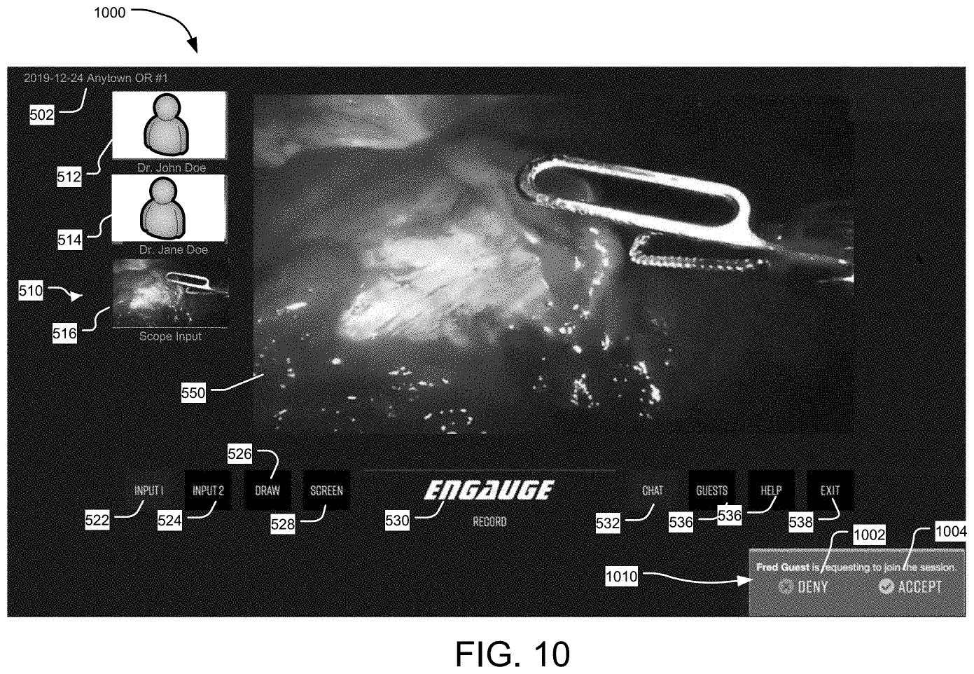

FIG. 10 depicts a guest announcement interface 1000 that may be presented on the session user interface 500 when a user that requires access permission to be given attempts to enter the session. In the case shown in FIG. 10, a user, “Fred Guest,” is attempting to enter the session. In turn, a guest announcement menu 1010 is presented. An authorized user with sufficient permissions to allow or deny guest users may be presented the guest announcement menu 1010. The guest announcement menu 1010 includes a deny guest button 1002 and an accept guest button 1004. Upon selection of the deny guest button 1002, the user is denied entry into the session and may receive a message indicating such. Upon selection of the accept guest button 1004, the user may be admitted to the session.

In turn, FIG. 11 shows a guest management interface 1100 that may be presented on the session user interface 500 upon selection of the guest management button 534. The guest management menu 1110 includes a listing of guests. In the example of FIG. 11, a first guest listing 1112 for Dr. John Doe, a second guest listing 1114 for Fred Guest, and a third guest listing 1116 for Dr. Jane Doe is shown. Because Dr. John Doe is the session administrator whose session user interface 500 is displayed, the first guest listing 1112 includes an indication of the identity of the user of the session user interface 500. Additionally, the first guest listing 1112 includes first guest information 1118 that includes the user's name and contact information. A user may select the first guest information 1118 to initiate a call or private chat with the user for the first guest information 1118. Similarly, the second guest listing 1114 includes second guest information 1120, and the third guest listing 1116 includes third guest information 1122.

Because Fred Guest is a guest user, the second guest listing 1114 also includes a guest removal button 1124 that allows an administrative user to remove the second guest from the session. In addition, a revocation of permission toggle 1126 is provided. If toggled, the revocation of permission toggle 1126 may revoke future access privileges of the second user. Because Dr. Jane Doe has sufficient permissions, a guest removal button 1124 is provided to allow for removal of the third user from the session, but no revocation of permission toggle 1126 is provided. A close button 1130 allows a user to return to the session user interface 500.

FIG. 12 shows a help interface 1200. The help interface 1200 may be shown on the session user interface 500 upon selection of the help button 536. The help interface 1200 includes a help menu 1210. The help menu 1210 may be used by a user to contact additional users or outside contacts for support. In this regard, the help menu 1210 includes a dial input 1212 that allows a phone number to be input to initiate a call within the session. This call may be recorded if the recording control button 530 has been toggled, and the session is being recorded. The help menu 1210 may also include a first pre-programmed contact 1214, a second pre-programmed contact 1216, a third pre-programmed contact 1218, and a fourth pre-programmed contact 1220. Additional pre-programmed contacts may also be provided. Selection of one of the first pre-programmed contact 1214, second pre-programmed contact 1216, third pre-programmed contact 1218, or fourth pre-programmed contact 1220 may initiate a call or request to join the session to a colleague at a respective one of the pre-programmed contacts. Furthermore, a technical support contact 1222 may be provided for contacting a platform representative for technical assistance with the session. A close button 1224 returns the user to the session user interface 500.

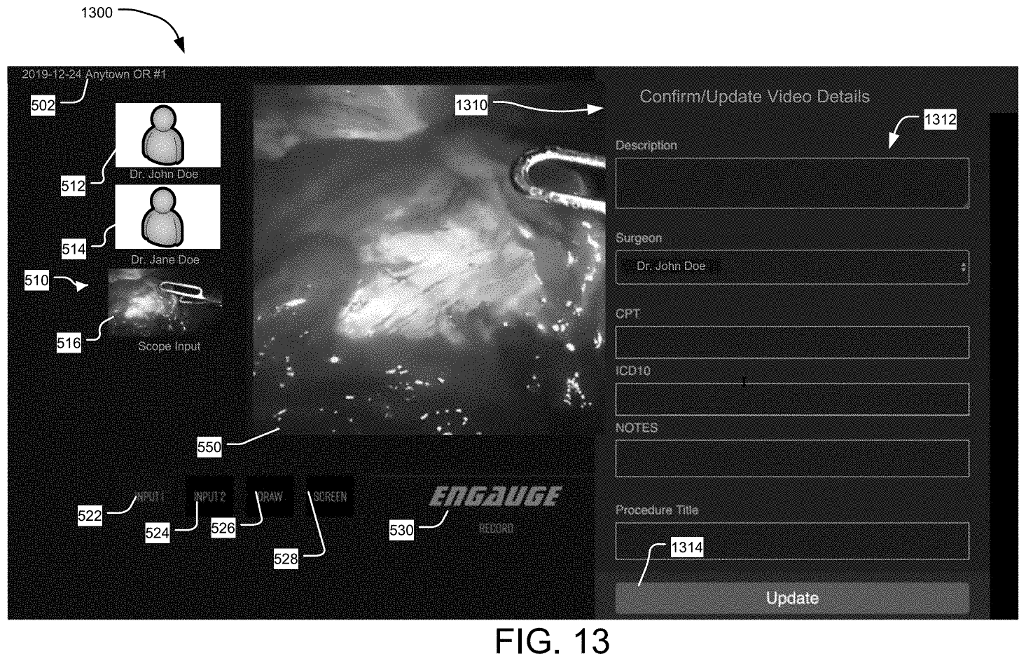

FIG. 13 shows a video metadata confirmation interface 1300. The video metadata confirmation interface 1300 may be displayed upon the completion of a recording when the recording control button 530 is toggled off. Upon completion of the recording, a metadata menu 1310 may be presented. The metadata menu 1310 includes metadata fields 1312. The metadata fields 1312 may include fields that may be populated with metadata. In at least some instances, the metadata fields 1312 may be prepopulated with information regarding the selection. In any regard, upon input and/or confirmation of the metadata in the metadata fields 1312, a confirmation button 1314 may be selected to save the metadata with the video. Further still, an administrator or other user may be capable of modifying the metadata fields provided for the surgical media. In this regard, a healthcare facility or provider may create a custom taxonomy at any appropriate granularity of metadata desired.

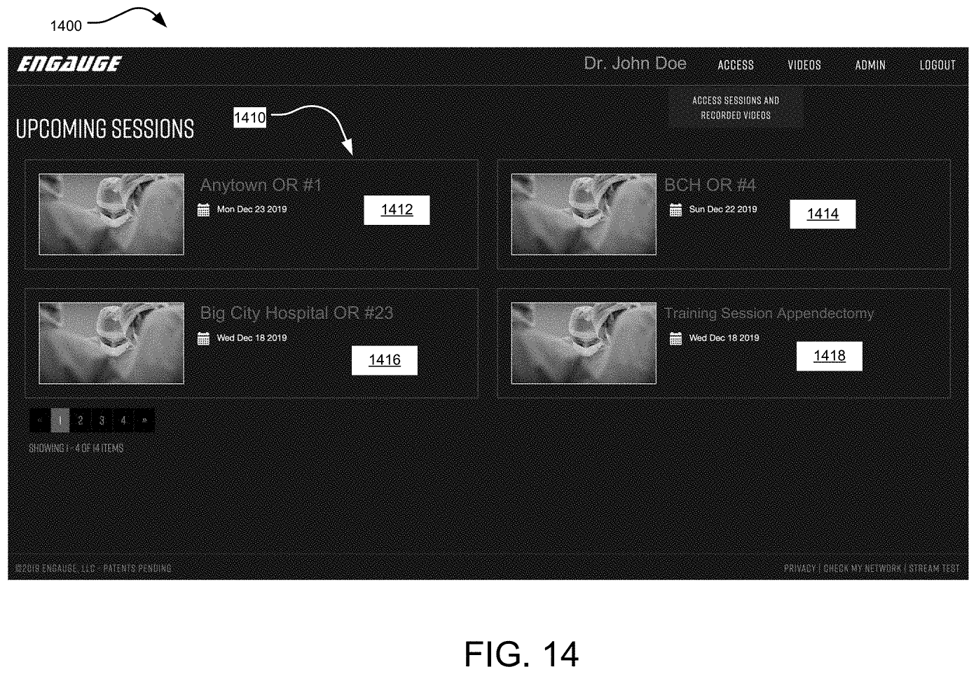

With further reference to FIGS. 14-17, various interfaces for the media access portal 426 from FIG. 4 are displayed that allows a user to see upcoming sessions, join a session, or view archived video from the session. In FIG. 14, a session listing interface 1400 is shown that lists upcoming sessions scheduled for a user. An upcoming session listing 1410 includes a first session 1412, a second session 1414, a third session 1416, and a fourth session 1418. Upon selection of any one of the sessions in the upcoming session listing 1410, a user may join the session.

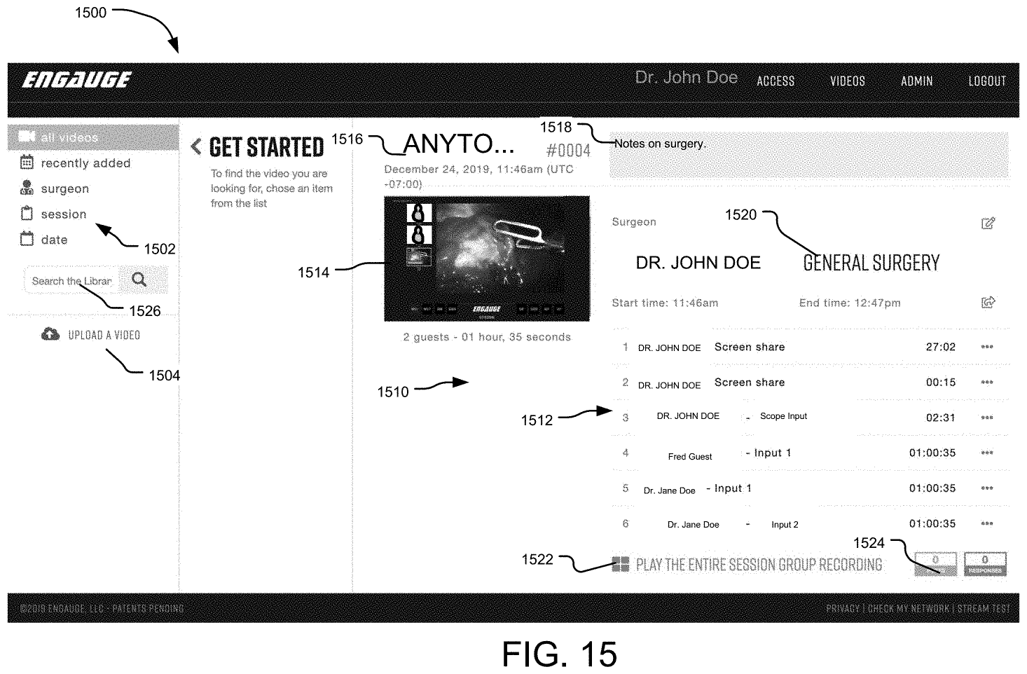

Once a video has been saved, the video may be later accessed from the cloud storage system of the surgical media management platform for playback by a user with proper credentials. For instance, FIG. 15 shows a video access interface 1500 that allows a user to search and select archival videos for playback. The video access interface 1500 includes a video search filters 1502 to allow a user to search for select videos. Also, a video search box 1526 may be provided for keyword searching. If a user wishes to upload a video to the example surgical media management platform (e.g., that was not recorded in the example surgical media management platform), a video upload selection 1504 is provided to upload a video file.

Once appropriately filtered or searched, videos may be represented for viewing as a video entry 1510 in a video listing. The video entry 1510 includes video details 1516, surgery notes 1518, and surgeon information 1520. Additionally, the video entry 1510 includes a source listing 1512 that shows what sources were included in the video of the video entry 1510 and at what times of the video. A group recording selection 1522 is provided to provide synched source playback of a video of a session, as will be described below. The video entry 1510 also includes social interaction indicators 1524 to show the number of posts, comments, and/or responses from the community regarding the video.

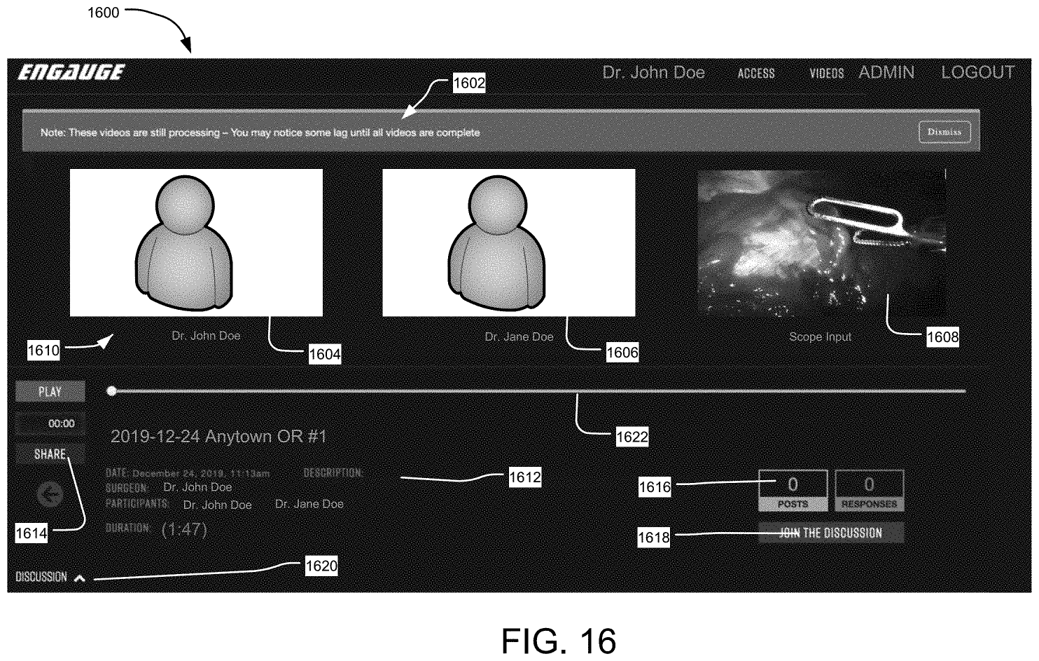

As noted in FIG. 15, a user may select a group recording selection 1522 from the video entry 1510. By doing so, a user is taken to a group recording playback interface 1600, as shown in FIG. 16. The group recording playback interface 1600 allows a user to view a synched replay of all sources that were recorded in the session. The group recording playback interface 1600 includes a video source array 1610 that presents the video feeds for the various sources of the session that are played back in a synched fashion to recreate the moment that was recorded in the session. A video conversion warning message 1602 may be provided if the conversion of the exchange surgical media data from the real-time transport format to the container format as described above is not complete. The video source array 1610 includes a first source video playback 1604, a second source video playback 1606, and a third source video playback 1608. As noted above, these video playbacks are synched to recreate the flow of the session that was recorded.

The group recording playback interface 1600 also includes a playback scrubber 1622 to control the playback of the synched video playbacks. Video details 1612 are provided for the video. Also, a share button 1614 is provided to easily allow a user to share a link or copy of the video with another party.

Social interaction indicators 1616 are provided to show what interaction has taken place regarding the video. For instance, a comment button 1618 is provided to allow a user to provide a comment regarding the video. In turn, comments, responses, and other posts are reflected in a discussion field 1620 for the video.

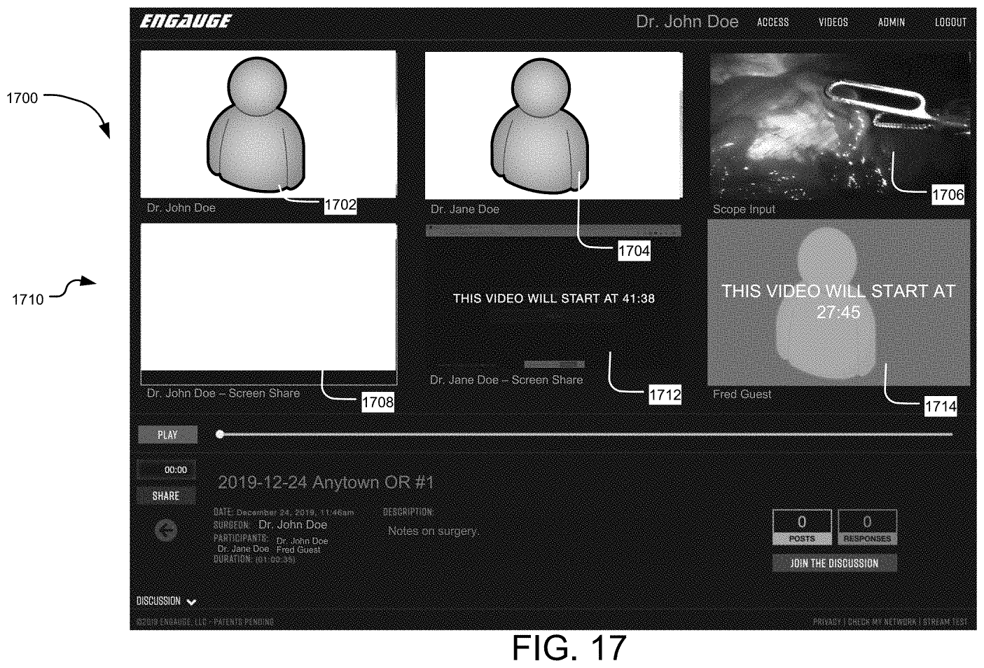

FIG. 17 shows a group recording playback interface 1700 in which not all sources of the video are present throughout the video. For instance, in group recording playback interface 1700 a video source array 1710 includes a first source video playback 1702, a second source video playback 1704, a third source video playback 1706, a fourth source video playback 1708, a fifth source video playback 1712, and a sixth source video playback 1714. The fifth source video playback 1712 includes a message that the source does not start until the 41 minute and 38 second mark of the video. Similarly, sixth source video playback 1714 includes a message that the sixth source does not become available until the 27 minute 45 second mark of the video. In this regard, the group recording playback interface 1700 includes a video source array 1710 that reflects the start time and/or availability of sources for the video.

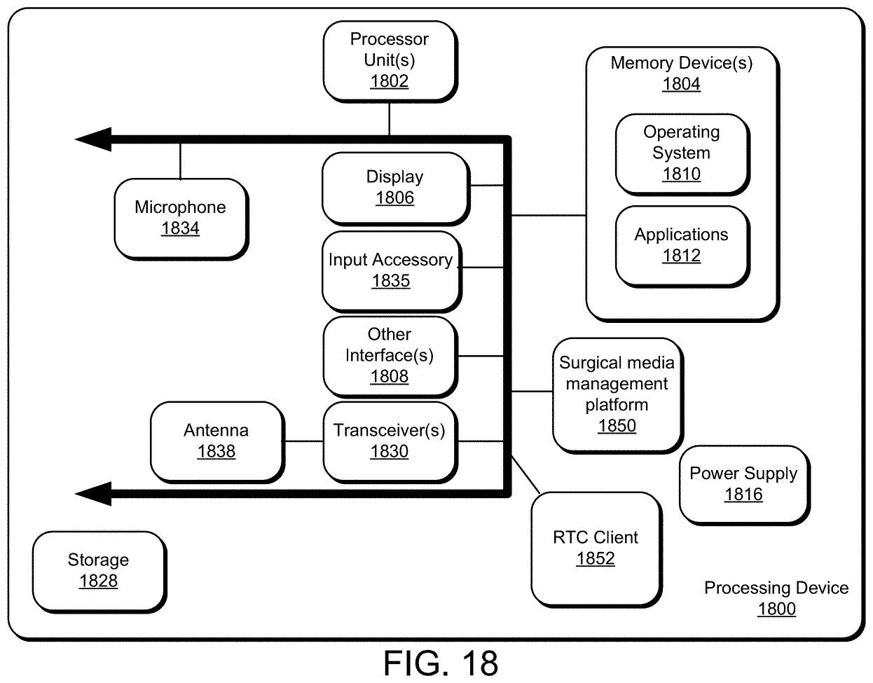

FIG. 18 illustrates an example schematic of a processing system 1800 suitable for implementing aspects of a surgical media management system as described above. For example, a processing system 1800 may be used to execute a surgical media management platform 1850. Additionally or alternatively, the source and/or participant devices executing a source RTC client 1852 or participant RTC client 1852 respectively may comprise processing systems 1800. Thus, while the processing system 1800 shows both a surgical media management platform 1850 and an RTC client 1852, the surgical media management platform 1850 may include one of a surgical media management platform 1850, an RTC client 1852, or both. The processing system 1800 includes one or more processor unit(s) 1802, memory 1804, a display 1806, and other interfaces 1808 (e.g., buttons). The memory 1804 generally includes both volatile memory (e.g., RAM) and non-volatile memory (e.g., flash memory). An operating system 1810, such as the Microsoft Windows® operating system, the Apple macOS operating system, or the Linux operating system, resides in the memory 1804 and is executed by the processor unit(s) 1802, although it should be understood that other operating systems may be employed.

One or more applications 1812 are loaded in the memory 1804 and executed on the operating system 1810 by the processor unit(s) 1802. Applications 1812 may receive input from various input local devices such as a microphone 1834, input accessory 1835 (e.g., keypad, mouse, stylus, touchpad, joystick, an instrument mounted input, or the like). Additionally, the applications 1812 may receive input from one or more remote devices such as remotely-located smart devices by communicating with such devices over a wired or wireless network using more communication transceivers 1830 and an antenna 1838 to provide network connectivity (e.g., a mobile phone network, Wi-Fi®, Bluetooth®). The processing device 1800 may also include various other components, such as a positioning system (e.g., a global positioning satellite transceiver), one or more accelerometers, one or more cameras, an audio interface (e.g., the microphone 1834, an audio amplifier and speaker and/or audio jack), and storage devices 1828. Other configurations may also be employed.

The processing device 1800 further includes a power supply 1816, which is powered by one or more batteries or other power sources and which provides power to other components of the processing device 1800. The power supply 1816 may also be connected to an external power source (not shown) that overrides or recharges the built-in batteries or other power sources.

In an example implementation, a surgical media management system and/or software embodied by instructions stored in the memory 1804 and/or the storage devices 1828 and processed by the processor unit(s) 1802. The memory 1804 may be the memory of a host device or of an accessory that couples to the host.

The processing system 800 may include a variety of tangible processor-readable storage media and intangible processor-readable communication signals. Tangible processor-readable storage can be embodied by any available media that can be accessed by the processing system 800 and includes both volatile and nonvolatile storage media, removable and non-removable storage media. Tangible processor-readable storage media excludes intangible communications signals and includes volatile and nonvolatile, removable and non-removable storage media implemented in any method or technology for storage of information such as processor-readable instructions, data structures, program modules or other data. Tangible processor-readable storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other tangible medium which can be used to store the desired information and which can be accessed by the processing system 800. In contrast to tangible processor-readable storage media, intangible processor-readable communication signals may embody processor-readable instructions, data structures, program modules or other data resident in a modulated data signal, such as a carrier wave or other signal transport mechanism. The term “modulated data signal” means an intangible communications signal that has one or more of its characteristics set or changed in such a manner as to encode information in the signal. By way of example, and not limitation, intangible communication signals include signals traveling through wired media such as a wired network or direct-wired connection, and wireless media such as acoustic, RF, infrared, and other wireless media.

Some implementations may comprise an article of manufacture. An article of manufacture may comprise a tangible storage medium to store logic. Examples of a storage medium may include one or more types of processor-readable storage media capable of storing electronic data, including volatile memory or non-volatile memory, removable or non-removable memory, erasable or non-erasable memory, writeable or re-writeable memory, and so forth. Examples of the logic may include various software elements, such as software components, programs, applications, computer programs, application programs, system programs, machine programs, operating system software, middleware, firmware, software modules, routines, subroutines, operation segments, methods, procedures, software interfaces, application program interfaces (API), instruction sets, computing code, computer code, code segments, computer code segments, words, values, symbols, or any combination thereof. In one implementation, for example, an article of manufacture may store executable computer program instructions that, when executed by a computer, cause the computer to perform methods and/or operations in accordance with the described implementations. The executable computer program instructions may include any suitable type of code, such as source code, compiled code, interpreted code, executable code, static code, dynamic code, and the like. The executable computer program instructions may be implemented according to a predefined computer language, manner or syntax, for instructing a computer to perform a certain operation segment. The instructions may be implemented using any suitable high-level, low-level, object-oriented, visual, compiled, and/or interpreted programming language.

One general aspect of the present disclosure includes a surgical media management system. The system includes a network-connected source computing device operative to receive surgical media data and a source real-time communication (RTC) client executing on the source computing device configured for encrypted communication of the surgical media data from the source computing device in real-time. The system also includes a cloud-hosted surgical media management platform operative to receive the encrypted surgical media data communicated from the source RTC client of the source computing device at least for storage of the surgical media data in a cloud storage instance. The system further includes a media access portal of the surgical media management platform accessible via network communications to authenticate remote access to the surgical media data in the cloud-hosted surgical media management platform.

Implementations may include one or more of the following features. For example, the system may include a media converter operative to receive surgical media data and convert the surgical media data into an input media format compatible with the source computing device. The source RTC client may include a source codec that encodes the surgical media data into a real-time transport format for communication to the cloud-hosted surgical media management platform.

In an example, the media access portal facilitates a live stream of the surgical media data in the real-time transport format with at least one participant RTC client executing on a participant computing device. The live stream may be facilitated by a peer to peer exchange of the surgical media data using a real-time protocol (RTP) mediated by the media access portal of the cloud-hosted surgical media management platform.

In an example, the cloud-hosted surgical media management platform further includes a platform codec to reformat the surgical media data from the real-time transport format to a container format for storage in the cloud storage instance. The media access portal may provide access to historical surgical media data for playback of the historical surgical media data in the container format.

In an example, the media access portal includes collaboration tools for real-time annotation of the surgical media data by a source user generating the surgical media data or a participant user remote from the source user via access to the media access portal. The media access portal may include user authentication for selective secure access to surgical media data in the cloud storage instance.

In an example, the surgical media data includes a plurality of media streams generated by a corresponding plurality of media sources.

In an example, the system further includes a health informatics interface of the surgical media management platform operative to receive healthcare data regarding the surgical media data. In turn, the surgical media management platform is operative to associate the healthcare data with the surgical media data as metadata. The system may further include an analytics engine of the surgical media management platform operative to apply an artificial intelligence approach to the healthcare data and the surgical media data to identify actionable intelligence from the surgical media data.

Another general aspect of the present invention includes a method for management of surgical media. The method includes receiving, in real-time, surgical media data at a cloud-hosted surgical media management platform from a source real-time communication (RTC) client of a source computing device. The source RTC client configured for communication of the surgical media data from the client computing device in real-time. The method also includes storing the surgical media data in a cloud storage instance. The method further includes hosting a media access portal of the surgical media management platform accessible via network communications to authenticate remote access to the surgical media data in the cloud-hosted surgical media management platform.

Implementations may include one or more of the following features. For example, the method may also include encoding, at a source codec of the source RTC client, the surgical media data from an input media format compatible with the source computing device to a real-time transport format for communication to the cloud-hosted surgical media management platform.

In an example, the method includes live streaming the surgical media data in the real-time transport format with at least one participant RTC client executing on a participant computing device using a real-time protocol (RTP) mediated by the media access portal of the cloud-hosted surgical media management platform. The method may also include reformatting the surgical media data from the real-time transport format to a container format for storage in the cloud storage instance. Furthermore, the method may include providing access to historical surgical media data for playback of the historical surgical media data in the container format.

In an example, the method includes providing collaboration tools for real-time annotation of the surgical media data by a source user generating the surgical media data or a participant user remote from the source user via access to the media access portal. In an example, the surgical media data includes a plurality of media streams generated by a corresponding plurality of media sources. The plurality of media streams are received and stored by the surgical media management platform.

In an example, the method includes receiving, at a health informatics interface of the surgical media management platform, healthcare data regarding the surgical media data and associating the healthcare data with the surgical media data as metadata in the cloud storage instance. In turn, the method may include applying an artificial intelligence approach to the healthcare data and the surgical media data to identify actionable intelligence from the surgical media data.

Another general aspect of the present disclosure includes one or more tangible processor-readable storage media embodied with instructions for executing on one or more processors and circuits of a device a process for management of surgical media. The process embodied in the one or more tangible processor-readable storage media includes receiving, in real-time, surgical media data at a cloud-hosted surgical media management platform from a source real-time communication (RTC) client of a source computing device, the source RTC client configured for communication of the surgical media data from the client computing device in real-time. The process also includes storing the surgical media data in a cloud storage instance and hosting a media access portal of the surgical media management platform accessible via network communications to authenticate remote access to the surgical media data in the cloud-hosted surgical media management platform.

While the foregoing examples have been illustrated and described in detail in the drawings and foregoing description, such illustration and description is to be considered as exemplary and not restrictive in character. The implementations described herein are implemented as logical steps in one or more computer systems. The logical operations may be implemented (1) as a sequence of processor-implemented steps executing in one or more computer systems and (2) as interconnected machine or circuit modules within one or more computer systems. The implementation is a matter of choice, dependent on the performance requirements of the computer system being utilized. Accordingly, the logical operations making up the implementations described herein are referred to variously as operations, steps, objects, or modules. Furthermore, it should be understood that logical operations may be performed in any order unless explicitly claimed otherwise or a specific order is inherently necessitated by the claim language.

What is claimed is:

1. A surgical media management system, comprising:
a network-connected source computing device operative to receive surgical media data;
a source real-time communication (RTC) client executing on the source computing device configured for encrypted communication of the surgical media data from the source computing device in real-time;
a cloud-hosted surgical media management platform operative to receive the encrypted surgical media data communicated from the source RTC client of the source computing device at least for storage of the surgical media data in a cloud storage instance; and
a media access portal of the surgical media management platform accessible via network communications to authenticate remote access to the surgical media data in the cloud-hosted surgical media management platform.

2. The system of claim 1, further comprising:
a media converter operative to receive surgical media data and convert the surgical media data into an input media format compatible with the source computing device, wherein the source RTC client comprises a source codec that encodes the surgical media data into a real-time transport format for communication to the cloud-hosted surgical media management platform.

3. The system of claim 2, wherein the media access portal facilitates a live stream of the surgical media data in the real-time transport format with at least one participant RTC client executing on a participant computing device.

4. The system of claim 3, wherein the live stream is facilitated by a peer to peer exchange of the surgical media data using a real-time protocol (RTP) mediated by the media access portal of the cloud-hosted surgical media management platform.

5. The system of claim 2, wherein the cloud-hosted surgical media management platform further comprises a platform codec to reformat the surgical media data from the real-time transport format to a container format for storage in the cloud storage instance.

6. The system of claim 5, wherein the media access portal provides access to historical surgical media data for playback of the historical surgical media data in the container format.

7. The system of claim 1, wherein the media access portal comprises collaboration tools for real-time annotation of the surgical media data by a source user generating the surgical media data or a participant user remote from the source user via access to the media access portal.

8. The system of claim 1, wherein the media access portal comprises user authentication for selective secure access to surgical media data in the cloud storage instance.

9. The system of claim 1, wherein the surgical media data comprises a plurality of media streams generated by a corresponding plurality of media sources.

10. The system of claim 1, further comprising:
a health informatics interface of the surgical media management platform operative to receive healthcare data regarding the surgical media data; and
wherein the surgical media management platform is operative to associate the healthcare data with the surgical media data as metadata.

11. The system of claim 10, further comprising:
an analytics engine of the surgical media management platform operative to apply an artificial intelligence approach to the healthcare data and the surgical media data to identify actionable intelligence from the surgical media data.

12. A method for management of surgical media, comprising:
receiving, in real-time, surgical media data at a cloud-hosted surgical media management platform from a source real-time communication (RTC) client of a source computing device, the source RTC client configured for communication of the surgical media data from the client computing device in real-time;
storing the surgical media data in a cloud storage instance; and
hosting a media access portal of the surgical media management platform accessible via network communications to authenticate remote access to the surgical media data in the cloud-hosted surgical media management platform.

13. The method of claim 12, further comprising:
encoding, at a source codec of the source RTC client, the surgical media data from an input media format compatible with the source computing device to a real-time transport format for communication to the cloud-hosted surgical media management platform.

14. The method of claim 13, further comprising:
live streaming the surgical media data in the real-time transport format with at least one participant RTC client executing on a participant computing device using a real-time protocol (RTP) mediated by the media access portal of the cloud-hosted surgical media management platform.

15. The method of claim 13, further comprising:
reformatting the surgical media data from the real-time transport format to a container format for storage in the cloud storage instance; and
providing access to historical surgical media data for playback of the historical surgical media data in the container format.

16. The method of claim 12, further comprising:
providing collaboration tools for real-time annotation of the surgical media data by a source user generating the surgical media data or a participant user remote from the source user via access to the media access portal.

17. The method of claim 12, wherein the surgical media data comprises a plurality of media streams generated by a corresponding plurality of media sources, the plurality of media streams being received and stored by the surgical media management platform.

18. The method of claim 12, further comprising:
receiving, at a health informatics interface of the surgical media management platform, healthcare data regarding the surgical media data; and
associating the healthcare data with the surgical media data as metadata in the cloud storage instance.

19. The method of claim 18, further comprising:
applying an artificial intelligence approach to the healthcare data and the surgical media data to identify actionable intelligence from the surgical media data.

20. One or more tangible processor-readable storage media embodied with instructions for executing on one or more processors and circuits of a device a process for management of surgical media, comprising:
receiving, in real-time, surgical media data at a cloud-hosted surgical media management platform from a source real-time communication (RTC) client of a source computing device, the source RTC client configured for communication of the surgical media data from the client computing device in real-time;
storing the surgical media data in a cloud storage instance; and
hosting a media access portal of the surgical media management platform accessible via network communications to authenticate remote access to the surgical media data in the cloud-hosted surgical media management platform.
